(19) United States
(12) Patent Application Publication
(10) Pub. No.: US 2026/0284449 A1
(43) Pub. Date: Sep. 24, 2026

(54) MASK AND A POWERED RESPIRATOR DEVICE

(71) Applicant: Chit Kwan Yau, New Territories (CN)

(72) Inventor: Chit Kwan Yau, New Territories (CN)

(21) Appl. No.: 19/690,461

(22) Filed: May 28, 2026

Related U.S. Application Data
(63) Continuation-in-part of application No. 18/700,040, filed on May 16, 2023.

(30) Foreign Application Priority Data
May 18, 2022 (CN) 202221190429.8

(51) Int. Cl. A62B 18/00; A61F 7/00; A62B 18/02; A62B 18/08; A62B 23/02

(52) U.S. Cl. CPC A62B 18/006; A61F 7/007; A62B 18/08; A62B 18/084; A62B 23/02; A61F 2007/0011; A61F 2007/0075; A62B 18/02

(57) ABSTRACT

A mask includes a frame, a light-transmitting sheet of convex shape, and a filter assembly; the frame covers a user's face and includes an opening directing to the user's lips. The light-transmitting sheet engages an edge of the first opening to cover it. Light inside the frame can be transmitted to the outside through the light-transmitting sheet; the filter assembly adapted to filter inhaled and exhaled air. Light transmitted through the light-transmitting sheet disperses in a fan shape, so the user's lip movements are observed without standing directly in front of the user. A powered respirator device includes: a wearable air supply unit including a blower, a controller and a filter, a conduit fluidly coupling the wearable air supply unit to a mask, the blower configured to draw ambient air through the filter and deliver a continuous flow of filtered air to the mask via the conduit.

### TECHNICAL FIELD

The present application relates to the technical field of air filtration, and in particular to a mask.

The present application also relates to a powered respirator device that provides filtered air to a mask.

### BACKGROUND

The statements herein merely provide background information related to the present disclosure, and do not necessarily constitute prior art. Melt-blown cloth is generally used as the filter element, and in order to ensure the filtering effect, the melt-blown cloth cover part of a face for filtering the air inhaled and exhaled by the mask user.

However, the mask is designed in such a way that other people will not be able to know the mouth shape of the mask's user. In some special scenarios, the mouth shape can be used as an important means of communication, such as the scenario in which the hearing impaired need to communicate with each other by reading lips through the mouth shape and the scenario in which it is difficult to communicate in a noisy environment and with difficult-to-distinguish voices.

In this regard, the patent No. CN215837224U discloses a mask, wherein the user is able to conveniently communicate with other people in the scenario by seeing lips through a light-transmitting member and an opening on the body of the mask opposite to the user's lips, thereby improving the efficiency of communication between the user and other people. However, the light transmissive sheet of this mask is in the shape of a flat plate, so that the lip movements of the user can only be observed when other people are directly in front of the user, and the visual range is small.

Additionally, traditional respiratory masks e.g., filtration masks, including N95 masks often cause significant discomfort due to heat buildup, moisture buildup and increased resistance to breathing for users. Additionally traditional respiratory masks can also cause pressure points or pressure sores, especially during prolonged use. This can lead to reduced compliance and lower work productivity. For individuals with respiratory conditions like asthma or allergic rhinitis, exposure to airborne irritants like PM2.5, pollen, or viruses can trigger severe health episodes. Existing effective masks can be difficult to tolerate for extended periods.

Current solutions like disposable N95s become costly for daily use and contribute to environmental waste. Industrial powered respirators are effective but are often too bulky and expensive for everyday personal or wider consumer use.

### TECHNICAL PROBLEM

It is one of the objects of embodiments of the present application to provide a mask intended to solve the problem that when the mask uses a flat plate-like light-transmitting member, the lip movements of the user can only be observed when another person is directly in front of the user.

It one of the objects of embodiments of the present application to provide a powered respirator device intended to solve the problem of providing continuous and reduced effort breathing or at least provide the public with a useful powered respirator device.

### TECHNICAL SOLUTION

In order to solve the above technical problem, the technical solution adopted in the embodiments of the present application is:

In a first aspect, there is provided a mask comprising: a first frame, a light transmissive sheet, and a filtration assembly; wherein the first frame is used to cover a face of a user; wherein the first frame comprises a first opening directing to lips of the user; wherein the light transmissive sheet engages an edge of first opening to cover the first opening, and the light on an inner side of the first frame is able to be projected to an outer side of the first frame through the light transmissive sheet; wherein the light transmissive sheet has a convex shape; wherein the filter assembly is adapted to filter inhaled and exhaled air; wherein the filter assembly comprises a base and a filter element; wherein the base is configured in an inner side of light transmissive sheet and comprises an air exchange channel inside; wherein the light transmissive sheet comprises air holes connected to the air exchange channel, and the filter element is installed inside the air exchange channel.

In one embodiment, the mask further comprises a soft cushion; the soft cushion is disposed on the inside of the first frame and is adapted to contact with a user's face; the soft cushion is disposed around the edge of the first opening.

In one embodiment, the mask further comprises a second frame; wherein the second frame is enclosed to form a second opening; the second frame is stacked on an outer side of the first frame, an outer edge of the first frame and an outer edge of the second frame are detachably connected by a buckle, the first opening is connected with the second opening, and the light-transmitting sheet is clamped between the first frame and the second frame.

In one embodiment, the first frame has a buckle seat at a left side edge and a right side edge thereof respectively. The positions of the second frame that is close to the left side edge and that is close to the right side edge have a buckle respectively, wherein the buckles are located on an inner side of the second frame, and the buckle and the buckle seat are adapted to be connected to each other.

In one embodiment, the mask further comprises a suspension assembly for hanging on a user's ears, head or neck; the suspension assembly is connected to the second frame.

In one embodiment, the second frame has an opening at a position close to the left side edge and a position close to the right side edge respectively; the suspension assembly comprises a belt, and the belt passes through the opening for connecting to the second frame; or the suspension assembly comprises a rope, and the rope passes through the opening for connecting to the second frame.

In one embodiment, the light-transmitting sheet comprises an anti-haze cover layer on a surface thereof.

In an embodiment, the mask comprises a plurality of filter assemblies, and the bases of the plurality of the filter assemblies are located in a compartment on an insider side of the light transmissive sheet.

In one embodiment, the filter element comprises a filter sponge or a filter sheet having an accordion-type structure.

In one embodiment, the base has a rectangular frame shape.

According to a further aspect, there is provided a powered respirator device comprising: a wearable air supply unit, the wearable air supply unit comprising a blower, a controller and a filter, wherein the wearable air supply unit is adapted to worn around a user's neck or on a user's shoulders, a conduit fluidly coupling the wearable air supply unit to a mask; and, wherein the blower is configured to draw ambient air through the filter and deliver a continuous flow of filtered air to the mask via the conduit to maintain a continuous positive pressure within the mask. The powered respirator device may be a personal powered respirator device.

In one embodiment the wearable air supply unit comprises a first housing and a second housing, the first and second housing being spaced apart from each other and connected by a bridging member, wherein the bridging member adapted to be suspended from a user's body to support the wearable air supply unit.

In one embodiment, the bridging member is a neck band adapted to be worn around a posterior aspect of a user's neck and wherein the first housing and second housing are configured to rest on a user's chest and/or shoulders to distribute wearable air supply unit's weight across the chest and/or shoulders and/or upper back.

In one embodiment, the neck band comprises a substantially U shape or an arcuate shape, and first housing and second housing are identical in size and shape such that the weight of the wearable air supply unit is evenly distributed.

In one embodiment the first housing contains a power supply, wherein the power supply is a battery and the second housing contains the blower and the filter, wherein the filter is a replaceable filter, and the power supply is electrically coupled to the blower to provide power to the blower, and the second housing being spaced away from the mask.

In one embodiment, the replaceable filter is a HEPA (High Efficiency Particulate Air) filter that is rated to at least a N95 level of filtration and the filter is upstream of the blower.

In one example, the respirator device comprises a mask adapted to form a seal over at least a user's nose and/or mouth of a user.

In one embodiment, the replaceable filter comprises a filter housing, a protection mesh, a pre-filter and a main filter, wherein the pre-filter is positioned between the protection mesh and the main filter, wherein the pre-filter is upstream of the main filter and the filter housing comprising a pair of actuatable clips adapted to removably clip to the second housing.

In one embodiment, at least the main filter is rated to at least a N95 level of filtration.

In one embodiment, the conduit is a flexible conduit, the conduit comprises a unit end connector to connect to the wearable air supply unit and a mask end connector adapted to connect to the mask, wherein the mask end connector comprises a connector housing, the connector housing defining a gas outlet opening, a sealing ring positioned around the gas outlet opening and a locking ring attached to the connector housing, wherein the locking ring is configured to releasably engage with a corresponding connector on the mask.

In one embodiment, the mask comprises a filtered mask inlet port, an exhalation port, and a tube docking connector integrated into the filtered mask inlet port to allow quick connection and disconnection of the mask end connector of the conduit, and wherein the tube docking connector comprises one or more engagement protrusions that are configured to engage with one or more engagement slots in the locking ring of the mask end connector of the conduit to form a connection between the mask and the conduit.

In one example, the mask comprises one of: a full face mask covering the eyes, nose and mouth, wherein the full face mask is configured to seal on a region of the face surrounding the eyes, nose and mouth, and the full face mask comprises a transparent visor, a disposable fabric face mask that is adapted to cover at least the nose and mouth of the user, or, a reusable elastomeric face mask that is adapted to cover at least the nose and mouth and the reusable face mask includes one or more vents formed in a body of the mask to vent exhaled gases; and, wherein each mask comprises the mask end connector.

In one embodiment, the wearable air supply unit comprises a thermoelectric cooling (TEC) device integrated into the bridging member, wherein the TEC device is configured to provide heat dissipation to a user and provide localized cooling.

In one embodiment, the TEC device is integrated into the neck band of the wearable air supply unit, wherein the TEC device is positioned to contact a posterior aspect of the user's neck in use, wherein the TEC device comprises: a thermoelectric substrate, a proximal surface adapted to be contact the user's skin in the posterior aspect of the neck, a distal surface exposed to ambient air; and, wherein the TEC device is electrically coupled to the battery, and wherein the TEC device (i.e., TEC module) is activated by power from the battery and configured to actively transfer heat from its proximal surface to its distal surface, thereby providing the localized cooling to the user's neck

In one embodiment, the respirator comprising at least one sensor operatively coupled to the controller and adapted to transmit a detected parameter to the controller, wherein the sensor is configured to detect at least one of an environmental parameter or a physiological parameter; and wherein the controller is programmed to modulate a speed of the blower in real-time based on data received from the at least one sensor to optimize filtration effectiveness and/or optimize power consumption of the power supply.

In one embodiment the controller is configured to control the blower based on real-time data received from the at least one environmental or physiological sensor, thereby optimizing at least one of filtration effectiveness, user comfort, or power consumption of the rechargeable power supply.

In one embodiment, the sensor comprises a pressure sensor adapted to measure pressure of the flow of gases from the blower, the pressure sensor configured to determine an inhalation phase or a breathing pattern of a user based on the measured pressure, and the controller further configured to control the blower to provide gases synchronized with the inhalation phase or breathing pattern of the user such that the flow is higher during an inhalation phase as compared to an exhalation phase.

In one embodiment, the sensor comprises a particulate sensor adapted to detect the ambient particulate matter, and wherein the controller is configured to: increase blower speed in response to an increase in detected ambient particulate matter or control the blower to a blower speed that is correlated to the measured ambient particulate matter.

In one embodiment, the mask comprises: a first frame for covering a user's face, wherein the first frame has a first opening opposite to the lips of the user, a light-transmitting sheet engaging an edge of the first opening to cover the first opening, such that light on an inner side of the first frame can be transmitted to an outer side of the first frame through the light-transmitting sheet, wherein the light-transmitting sheet is convex, the base is arranged on the inner side of the light-transmitting sheet and is a has a ventilation channel internally, the light-transmitting sheet comprises air holes in communication with the ventilation channel.

In an embodiment the powered respirator device comprises a wireless connectivity module e.g., a Bluetooth module or a Cellular module, configured to transmit system performance data and filter life status to a mobile device.

According to a further aspect, there is provided a powered respirator device for providing filtered air comprising: a wearable air supply unit, the wearable air supply unit comprising a blower, a controller and a filter, wherein the wearable air supply unit is adapted to worn around a user's neck or on a user's shoulders, a mask comprising: a first frame for covering a user's face, wherein the first frame has a first opening opposite to the lips of the user; a light-transmitting sheet engaging an edge of the first opening to cover the first opening, such that light on an inner side of the first frame can be transmitted to an outer side of the first frame through the light-transmitting sheet, wherein the light-transmitting sheet is convex; and a filter assembly for filtering inhaled and exhaled air, wherein the filter assembly comprises a base and a filter element, the base is arranged on the inner side of the light-transmitting sheet and is a has a ventilation channel internally, the light-transmitting sheet comprises air holes in communication with the ventilation channel, and the filter element is installed in the ventilation channel, a conduit fluidly coupling the wearable air supply unit to the mask; and, wherein the blower is configured to draw ambient air through the filter and deliver a continuous flow of filtered air to the mask via the conduit to maintain a continuous positive pressure within the mask, and wherein the filter is a replaceable filter.

In one embodiment, wherein the wearable air supply unit comprises a first housing and a second housing, the first and second housing being spaced apart from each other and connected by a bridging member, wherein the bridging member adapted to be suspended from a user's body to support the wearable air supply unit. wherein the bridging member is a neck band adapted to be worn around a posterior aspect of a user's neck and wherein the first housing and second housing are configured to rest on a user's chest and/or shoulders to distribute wearable air supply unit's weight across the chest and/or shoulders and/or upper back, wherein the neck band comprises a substantially U shape or an arcuate shape, and first housing and second housing are identical in size and shape such that the weight of the wearable air supply unit is evenly distributed, wherein the first housing contains a power supply, wherein the power supply is a battery and the second housing contains the blower and the filter, wherein the filter is a replaceable filter, and the power supply is electrically coupled to the blower to provide power to the blower, and the second housing being spaced away from the mask; and, wherein the replaceable filter is a HEPA (High Efficiency Particulate Air) filter that is rated to at least a N95 level of filtration and the filter is upstream of the blower.

In one embodiment, wherein the conduit is a flexible conduit, the conduit comprises a unit end connector to connect to the wearable air supply unit and a mask end connector adapted to connect to the mask, wherein the mask end connector comprises a connector housing, the connector housing defining a gas outlet opening, a sealing ring positioned around the gas outlet opening and a locking ring attached to the connector housing, wherein the locking ring is configured to releasably engage with a corresponding connector on the mask.

In one embodiment, the respirator comprises at least one sensor operatively coupled to the controller and adapted to transmit a detected parameter to the controller, wherein the wearable air supply unit comprises a thermoelectric cooling (TEC) device integrated into the bridging member, wherein the TEC device is configured to provide heat dissipation to a user and provide localized cooling, wherein the TEC device is integrated into the neck band of the wearable air supply unit, wherein the TEC device is positioned to contact a posterior aspect of the user's neck in use, wherein the TEC device comprises: a thermoelectric substrate, a proximal surface adapted to be contact the user's skin in the posterior aspect of the neck, a distal surface exposed to ambient air, wherein the TEC device is electrically coupled to the battery, and wherein the TEC device (i.e. TEC module) is activated by power from the battery and configured to actively transfer heat from its proximal surface to its distal surface, thereby providing the localized cooling to the user's neck, the at least one sensor is a pressure sensor and/or a particulate sensor, wherein the pressure sensor is adapted to measure pressure of the flow of gases from the blower, the pressure sensor configured to determine an inhalation phase or a breathing pattern of a user based on the measured pressure, and the controller further configured to control the blower to provide gases synchronized with the inhalation phase or breathing pattern of the user such that the flow is higher during an inhalation phase as compared to an exhalation phase; and, wherein the particulate is adapted to detect the ambient particulate matter, and, wherein the controller is configured to: increase blower speed in response to an increase in detected ambient particulate matter or control the blower to a blower speed that is correlated to the measured ambient particulate matter.

According to a further aspect, there is provided a wearable air supply unit for a personal respirator, comprising: a first module and a second module connected by a neckband, the modules configured to be suspended from a user's neck and rest on a user's clavicle area, the first module containing a battery, the second module containing a motor-driven blower and a replaceable filter; and, at least one thermoelectric cooling (TEC) module disposed within the neckband, the TEC module having a first surface in thermal communication with a portion of the neckband intended to contact a user's skin and a second surface configured to dissipate heat.

According to a further aspect, there is provided a powered respirator device, comprising: a facepiece configured to form a seal over at least a user's nose and mouth, a wearable air supply unit configured to be worn around a user's neck, the wearable air supply unit comprising a first housing and a second housing interconnected by a neckband, an electric blower disposed within one of the first or second housings, the electric blower configured to draw ambient air through a filter and actively deliver a continuous flow of filtered air to the facepiece at a flow rate sufficient to create a positive pressure differential within the facepiece to reduce inhalation resistance for the user; and, a flexible air conduit fluidly connecting the wearable air supply unit to the facepiece.

In an embodiment, the facepiece comprises a dedicated filtered air inlet port for receiving the conduit and at least one exhalation valve configured to exhaust carbon dioxide. The powered respirator device may be adapted to function as a personal respiratory protection system that is adapted to provide breathable gases e.g., air to the user and protect the user's respiratory system by providing filtered breathable gases e.g., filtered air.

In an embodiment, the first housing and the second housing are configured to rest on a user's chest or shoulders to distribute the weight of the system away from the user's face.

In an embodiment, the first housing contains a power supply, and the second housing contains the electric blower and a replaceable filter assembly. The replaceable filter assembly is a High-Efficiency Particulate Air (HEPA) filter rated for at least N95-level filtration.

According to a further aspect, there is provided a smart personal respiratory protection system, comprising: a facepiece (i.e., a mask), a neck-worn air supply unit comprising a blower, a filter, and a controller, a conduit fluidly connecting the neck-worn air supply unit to the facepiece (i.e. mask); and, at least one sensor communicatively coupled to the controller, the sensor configured to detect at least one of an environmental parameter or a physiological parameter, wherein the controller is programmed to modulate an operational speed of the blower in real-time based on data received from the at least one sensor to optimize filtered air delivery.

In one embodiment, the at least one sensor comprises a first pressure port positioned to detect ambient pressure and a second pressure port positioned to detect pressure at an outlet of the blower.

In one embodiment, the controller calculates a differential pressure to determine a breathing rate of the user and adjusts the blower speed to synchronize air delivery with the user's inhalation. In one embodiment, the controller comprises a wireless connectivity module configured to transmit system performance data and filter life status to a mobile device

According to a further aspect, there is provided wearable air supply unit for a personal respirator, comprising: a first housing containing a power source, a second housing containing a blower and a filter, and a neckband interconnecting the first and second housings, wherein the neckband configured to suspend the housings from a user's neck such that the weight is distributed across the user's clavicle area, wherein the second housing includes a filtered air discharge port configured for fluid connection to a flexible conduit; and, wherein the filter is a removable filter that may be removably attachable to the second housing, the filter being positioned upstream of the blower, and the filter filter is a HEPA (High Efficiency Particulate Air) filter that is rated to at least a N95 level of filtration.

According to a further aspect, there is provided a replaceable filter that comprises a filter housing, a protection mesh, a pre-filter and a main filter, wherein the pre-filter is positioned between the protection mesh and the main filter, wherein the pre-filter is upstream of the main filter and the filter housing comprising a pair of actuatable clips adapted to removably clip to the second housing, wherein the main filter is a HEPA filter. The HEPA filter may be rated to a N95 level of filtration. Optionally, the pre-filter may be a coarse filter and the main filter may be a more fine filter. In one example, the protection mesh is configured to protect the pre-filter and main filter from large debris.

### BENEFICIAL EFFECTS

In one embodiment of the present application, there is provided a mask that has the beneficial effects as follows: the light-transmitting sheet of the present embodiment being convex in shape, so that the light transmitted through the light-transmitting sheet can be scattered in a fan shape, and other people can see the lip movements of the user without standing in front of the user, thereby improving the communication efficiency between the user and other people. In addition, by arranging the light-transmitting sheet as the outer convex sheet-shaped structure, the overall thickness and weight of the light-transmitting sheet can also be reduced, thereby facilitating sound transmission.

In one embodiment of the present application, there is provided a powered respirator device that comprising: a wearable air supply unit, the wearable air supply unit comprising a blower, a controller and a filter, wherein the wearable air supply unit is adapted to worn around a user's neck or on a user's shoulders, a conduit fluidly coupling the wearable air supply unit to a mask; and, wherein the blower is configured to draw ambient air through the filter and deliver a continuous flow of filtered air to the mask via the conduit to maintain a continuous positive pressure within the mask. The filter may be a HEPA (High Efficiency Particulate Air) filter that is rated to at least a N95 level of filtration and the filter is upstream of the blower. The respirator device has a lightweight and ergonomic design with the fan/filter situated away from the face for better heat dissipation and user comfort. The powered respirator device also provides continuous, effortless breathing with filtered air making it easier for a user to breathe and provides filtered air. The respirator device may also comprise a heat dissipation unit e.g., a TEC device to provide localized cooling to a user's skin.

### BRIEF DESCRIPTION OF THE DRAWINGS

In order to more clearly illustrate the technical solutions in the embodiments of this application, the accompanying drawings of the embodiments are provided, or the exemplary technical descriptions are briefly described below. Obviously, the accompanying drawings in the following description merely represent some embodiments of this application. A person of ordinary skilled in the art may derive other accompanying drawings according to these accompanying drawings without creative efforts.

FIG. 1 is a diagram of a mask according to an embodiment of this application;

FIG. 2 is another diagram of a mask according to an embodiment of this application;

FIG. 3 is an exploded view diagram of a mask according to an embodiment of this application;

FIG. 4 is a cross-sectional view diagram of a mask according to an embodiment of this application;

FIG. 5 is a vertical sectional view diagram of a mask according to an embodiment of this application;

FIG. 6 is a structural diagram of a light transmissive sheet and a filter assembly of a mask according to an embodiment of this application;

FIG. 7 is another structural diagram of a light transmissive sheet and a filter assembly of a mask according to an embodiment of this application;

FIG. 8 is a vertical sectional view diagram of the filter assembly shown in FIGS. 6 and 7;

FIG. 9 is a structural diagram of a light transmissive sheet of a mask and another filter assembly according to an embodiment of this application;

FIG. 10 is another structural diagram of a light transmissive sheet and another filter assembly of a mask according to an embodiment of this application;

FIG. 11 is a vertical sectional view of the filter assembly shown in FIGS. 9 and 10;

FIG. 12 is a structural diagram of a second frame of a mask and a suspension assembly according to an embodiment of this application;

FIG. 13 is a structural diagram of a second frame and another suspension assembly of a mask according to an embodiment of this application;

FIG. 14 is a structural diagram of a second frame of a mask and another suspension assembly according to an embodiment of this application;

FIG. 15 is a structural diagram of a mask including a one-way valve according to an embodiment of this application;

FIG. 16 is a schematic structural diagram of the light-transmitting sheet in FIG. 15;

FIG. 17 is a schematic structural diagram of the one-way valve in FIG. 15;

FIG. 18 is a schematic cross-sectional view diagram of the one-way valve in FIG. 15;

FIG. 19 is an exploded view diagram of the one-way valve in FIG. 17;

FIG. 20 is a schematic structural diagram of the base in FIG. 17;

FIG. 21 is a schematic structural diagram of the outer cover in FIG. 17.

FIG. 22 and FIG. 23 illustrate a front perspective view and a rear perspective view of a powered respirator device.

FIG. 24 illustrates removable and replaceable consumable modules of the powered respirator device.

FIG. 25 and FIG. 26 illustrates details of the replaceable filter.

FIG. 27 an example of the flexible conduit and its parts.

FIG. 28 illustrates details of the mask end connector of the flexible conduit.

FIG. 29 illustrates an example of a disposable mask used as part of the powered respirator device.

FIG. 30 illustrates an example of a replaceable elastomeric mask used as part of the powered respirator device.

FIG. 31 illustrates an example of a full-face mask (total face mask) used as part of the powered respirator device.

FIG. 32A illustrates the internal components of the wearable air supply unit. FIG. 32B illustrates another example of the powered respirator device with a TEC cooling unit. FIG. 32C shows the same powered respirator device as shown in FIG. 32B. FIG. 32D shows the powered respirator device of FIG. 32C, from an alternative viewing angle.

FIG. 33 illustrates an example operation of the powered respirator device; and FIG. 34 illustrates a block diagram of the electric and pneumatic architecture of the powered respirator device of FIG. 1.

In the drawings, 100, the first frame; 110, the first opening; 120, the buckle seat; 200, the light-transmitting sheet; 210, the air holes; 220, the air outlet;

300 filter assembly; 310, base; 311, plate; 312, cover; 320, filter element;

400 a soft cushion;

500 second frame; 510, second opening; 520, buckle; 530, through hole;

600 suspension assembly; 610, belt; 620, rope; 630, adjusting buckle;

700 one-way valve; 710, base; 711, bottom plate; 712, first convex ring; 713, mounting plate; 7131, first opening; 7132, connecting strip; 714, mounting column; 715, first convex rib; 720, outer cover; 721, cover plate; 7211, second through groove; 722, second convex ring; 723, second convex rib; 730, diaphragm; 740, valve cavity.

### EMBODIMENTS OF THE INVENTION

In order to clarify the objectives, technical solutions, and advantages of this application, there is provided the following descriptions of this invention in details with reference to the accompanying drawings and embodiments. It should be understood that the specific embodiments described herein are only used to explain the present disclosure and are not intended to limit the present disclosure.

It should be noted that when a component is referred to as being “affixed to” or “disposed on” another component, it may be directly on or indirectly on another component. When a component is referred to as being “connected to” another component, it may be directly or indirectly connected to the other component. The orientation or positional relationship indicated by the terms “upper”, “lower”, “left”, “right” and the like is only for ease of description, rather than indicating or implying that the referred device or element must have a specific orientation, and is constructed and operated in a specific orientation, and therefore the orientation or positional relationship cannot be understood as a limitation to the present application, and a person of ordinary skill in the art may understand the specific meanings of the above terms according to specific situations. The terms “first” and “second” are only used for convenience of description and cannot be understood as indicating or implying relative importance or implicitly indicating the number of technical features. The meaning of “a plurality of” is two or more unless specifically defined otherwise.

To illustrate the technical solutions of the present application, the following provides detailed descriptions with references to specific accompanying drawings and embodiments.

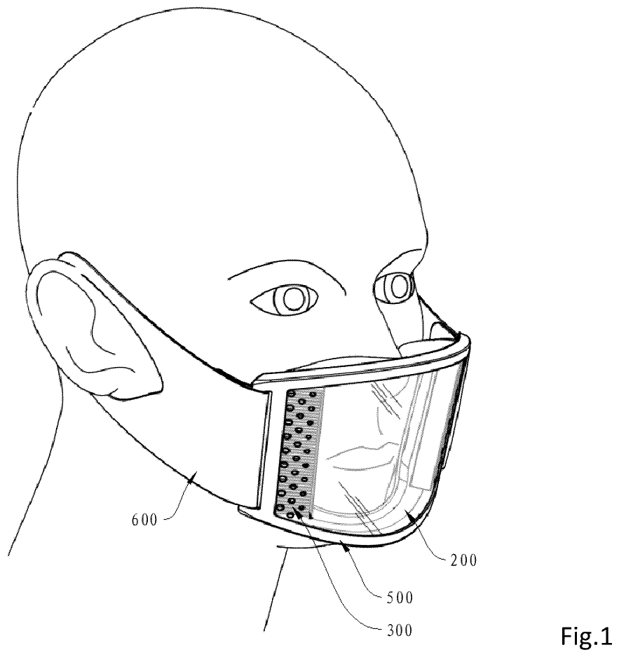

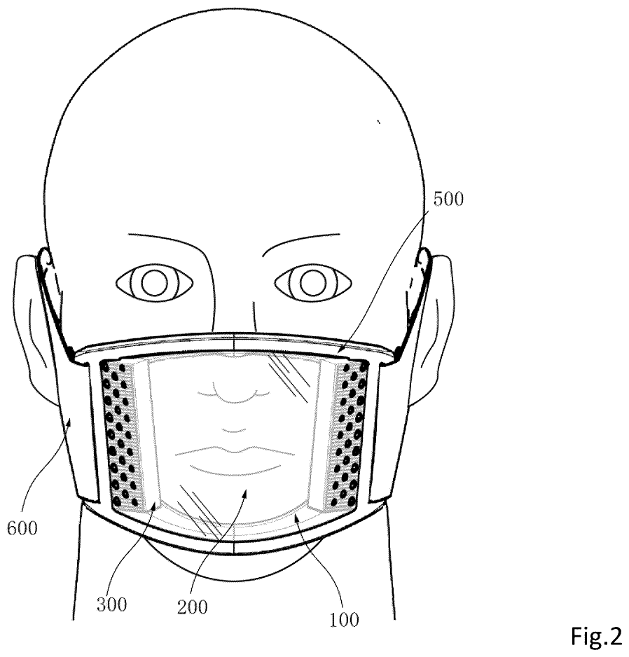

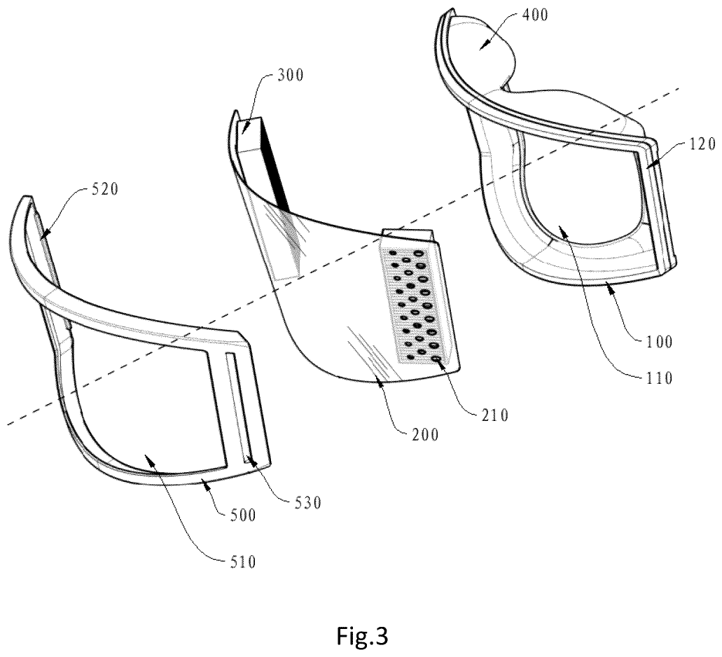

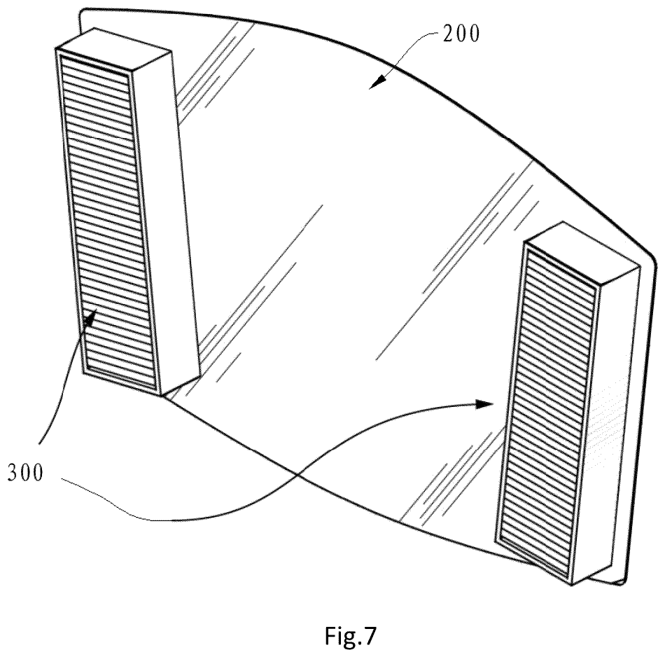

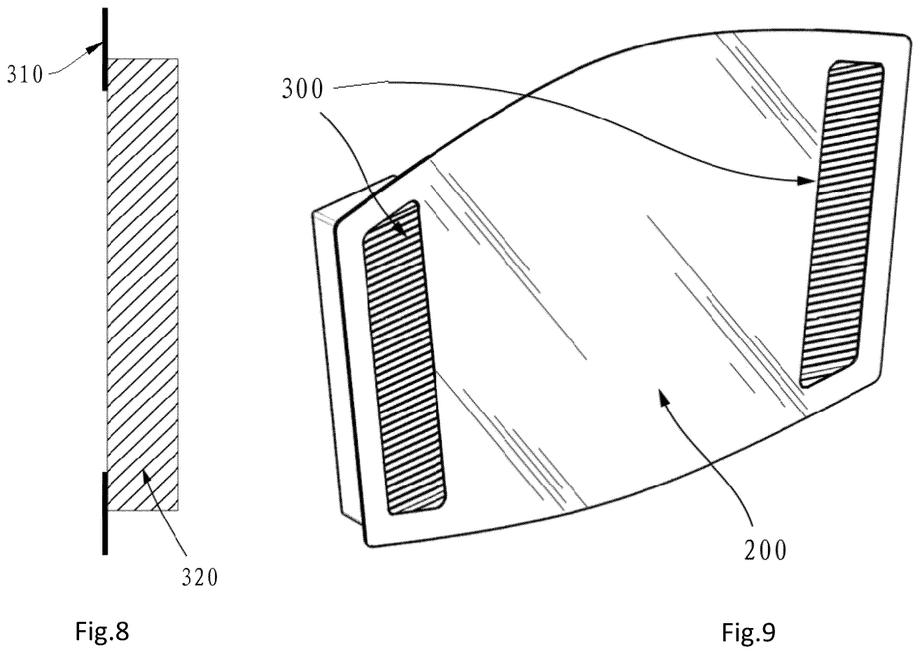

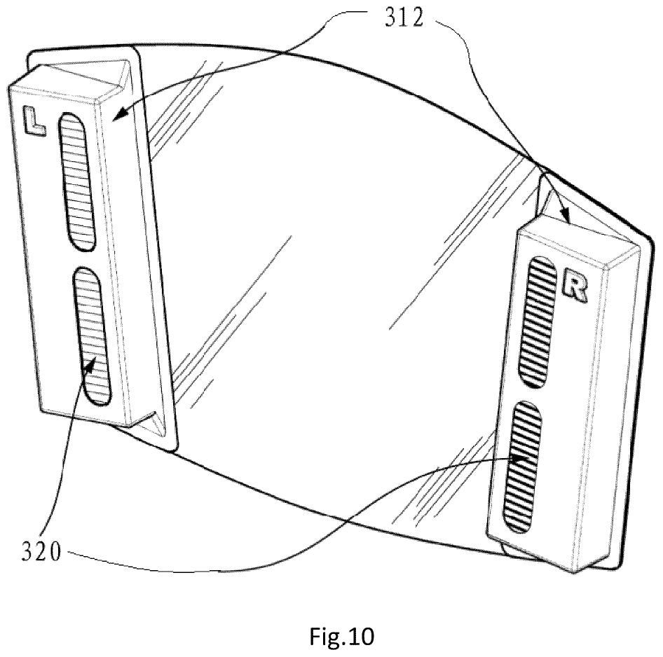

Referring to FIG. 1 to FIG. 13, there is provides a mask according to a preferred embodiment of this application comprising a first frame 100, a light-transmitting sheet 200, and a filter assembly 300. The first frame 100 is used for covering a face of the user. The first frame 100 comprises a first opening 110 opposite to the lips of a user. The light transmitting sheet 200 engages the edge of the first opening 110 to cover the first opening 110, such that light on an inner side of the first frame 100 can be transmitted to an outer side of the first frame 100 through the light transmitting sheet 200. The light transmitting sheet 200 has a convex shape. The filtering assembly 300 is adapted to filtering air inhaled and exhaled. The filtering assembly 300 comprises a base 310 and a filter element 320. The base 310 is arranged on the inner side of the light transmitting sheet 200 and comprises a ventilation channel internally. The light-transmitting sheet 200 has air holes 210 in communication with the ventilation channel. The filter element 320 is installed inside the ventilation channel.

When the mask provided in this embodiment is in use, the first frame 100 is covered on the face of the user, so that the first frame 100 along with the light-transmitting sheet 200 and the face of the user form an enclosed space such that the lips and nose of the user are within the enclosed space. During inhalation, air enters the ventilation channel through the air holes 210 on the light-transmitting sheets 200, and enters the enclosed space after being filtered by the filter element 320 inside the ventilation channel for the user to inhale. The gas exhaled by the user enters the enclosed space and passes through the ventilation channel, and after being filtered by the filter element 320 inside the ventilation channel, the gas exits out from the air hole 210 on the light-transmitting sheet 200, so that the user completes one complete breathing cycle.

When in use, since the first opening 110 is opposite to the lips of the user, the light can be transmitted out of the first opening 110 and through the light-transmitting sheet 200, such that other people can see the lips movement of the user.

Compared with the related prior art, the light-transmitting sheet 200 of the present embodiment is convex in shape, so that the light transmitted through the light-transmitting sheet 200 can be scattered in a fan shape, and other people can observe the lip movement of the user without standing in front of the user, thereby improving the communication efficiency between the user and other people. In addition, by designing the light-transmitting sheet 200 as a convex shaped structure, the overall thickness and weight of the light-transmitting sheet 200 can also be reduced, thereby facilitating sound transmission.

Optionally, the light-transmitting sheet 200 can be rectangular. Optionally, the light-transmitting sheet 200 may also have other shapes such as a circle, a three-dimensional shape, and the like.

Optionally, the light-transmitting sheet 200 can be made as follows: when the user wears the mask of the embodiment on the face of the user, the light-transmitting sheet 200 is inclined downwards. Firstly, in this configuration, the top end position of the mask can be lowered and will not affect the position when the user wearing glasses. Secondly, this configuration can reduce the reflection of light that obstructs others from seeing the face of the user. Thirdly, the visual angle below the eyes of the user will be increased.

Optionally, decorations may be added to the mask as three-dimensional components or printed on the planar surface of the light-transmitting sheet 200.

In some embodiments, referring to FIG. 1 to FIG. 13, the mask further comprises a soft cushion 400. The soft cushion 400 is disposed on an inner side of the first frame 100 and is configured to be in contact with a user's face. The soft cushion 400 is disposed around an edge of the first opening 110. In this embodiment, with the configuration of the soft cushion 400, the mask will not exert excessive pressure on the face of the user to improve the comfort of the mask. Preferably, the shape of the soft cushion 400 is complementary to the face of the user, for example, the soft cushion 400 is provided with a groove that is complementary to the nose bridge, so as to further improve the wearing comfort for the user.

In some embodiments, referring to FIG. 1 to FIG. 13, the mast further comprises a second frame 500. The second frame 500 comprises a second opening 510. The second frame 500 is adapted to be stacked on an outer side of the first frame 100. An outer edge of the first frame 100 and an outer edge of the second frame 500 are detachably connected by a buckle. The first opening 110 is in communication with the second opening 510. The light-transmitting sheet 200 is clamped between the first frame 100 and the second frame 500. In this embodiment, the light-transmitting sheet 200 is clamped between the first frame 100 and the second frame 500. On one hand, the light-transmitting sheet 200 can be securely mounted on the mask. On the other hand, the first frame 100 and the second frame 500 which are detachably connected by means of a buckle can be detached to remove the light-transmitting sheet 200. The filtering assembly 300 is installed in an inner side of the light-transmitting sheet 200, so that the light-transmitting sheet 200 and the filtering assembly 300 can be replaced, and thereby extending the service life of the mask of the present application.

Preferably, the light-transmitting sheet 200 is made with an elastic sheet, which can be deformed easily but is not easily damaged by the first frame 100 and the second frame 500.

In some embodiments, referring to FIG. 1 to FIG. 13, a left side edge and a right side edge of the first frame 100 are attached to a buckle seat 120 respectively. A position of the second frame 500 close to the left side edge and a position close to the right side edge are attached to a buckle 520 respectively. The buckle 520 is located on an inner side of the second frame 500. The buckle 520 and the buckle seat 120 are adapted to connect to each other. In this embodiment, the left and right sides of the first frame 100 and the second frame 500 are adapted to detachably secure to each other.

In other embodiments, the buckle seat 120 is arranged on the upper and lower side edges of the first frame 100, and the buckle 520 is provided on the upper and lower side edges of the second frame 500, so that the first frame 100 and the second frame 500 are detachably secured to each other.

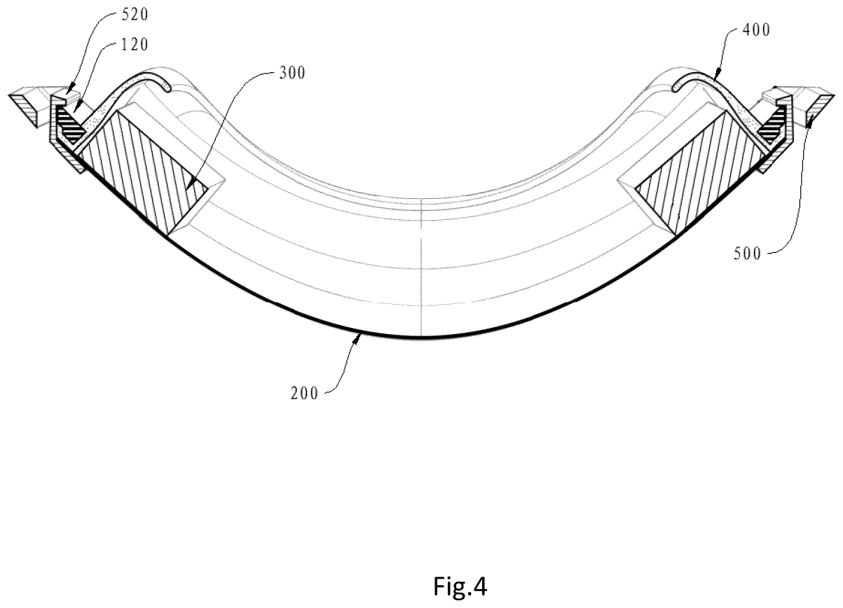

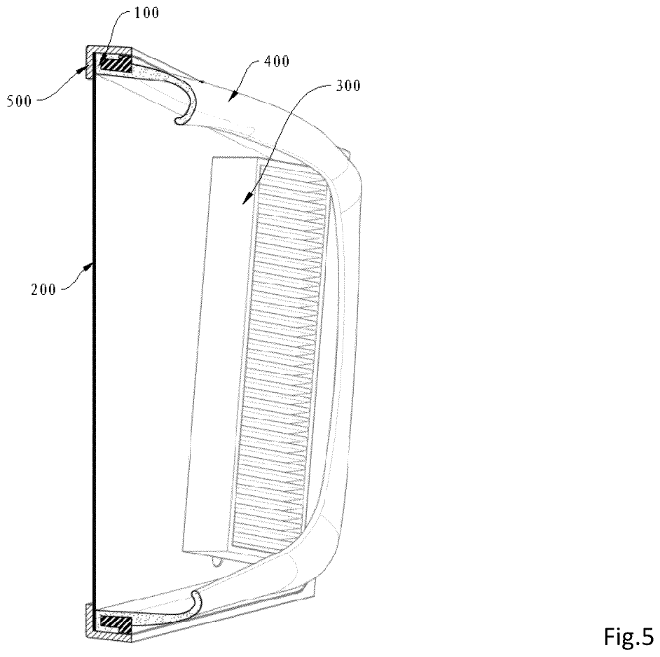

In some embodiments, referring to FIG. 4 and FIG. 5, a peripheral edge of the soft cushion 400 is clamped between a peripheral edge of the first frame 100 and a peripheral edge of the second frame 500. Specifically, a peripheral edge of the soft cushion 400 is first stacked on an outer side of a peripheral edge of the first frame 100, then a peripheral edge of the light-transmitting sheet 200 is stacked on an outer side of a peripheral edge of the soft cushion 400. Then a peripheral edge of the second frame 500 is stacked on an outer side of a peripheral edge of the light-transmitting sheet 200. Finally, the soft cushion 400 and the light-transmitting sheet 200 engage the first frame 100 and the second frame 500 such that the first frame 100, the soft cushion 400, the light-transmitting sheet 200 and the second frame 500 can be sealingly connected together.

In some embodiments, referring to FIG. 4 and FIG. 5, the buckle 520 extends inwards from a peripheral edge of the second frame 500 to the inner side of the buckle base 120. In other words, the light-transmitting piece 200, the soft cushion 400 and the first frame 100 are clamped between the buckle 520 and a peripheral edge of the second frame 500 in that order, thereby ensuring that the first frame 100, the soft cushion 400, the light-transmitting piece 200 and the second frame 500 are sealingly connected together. Since the buckle 520 is elastic, the soft cushion 400, the light-transmitting piece 200 or the filter assembly 300 can be detached for replacement only by pulling the buckle 520 away from the buckle base 120.

In some embodiments, referring to FIG. 4 and FIG. 5, a groove is formed on the outer side wall of the soft cushion 400. The first frame 100 is at least partially clamped in the groove. In one aspect, the soft cushion 400 can be affixed to the first frame 100. In another aspect, this can improve the sealing between the first frame 100 and the soft cushion 400.

In some embodiments, referring to FIG. 1 to FIG. 13, the mask further comprises a suspension assembly 600 for hanging on a user′ ears, and head or neck. The suspension assembly 600 is connected to the second frame 500, so that when wearing, the suspension assembly 600 exerts a force on the second frame 500 in the direction towards the first frame 100. In this way, the light-transmitting sheet 200 can be securely clamped between the first frame 100 and the second frame 500. In other embodiments, the suspension assembly 600 may also be connected to the first frame 100.

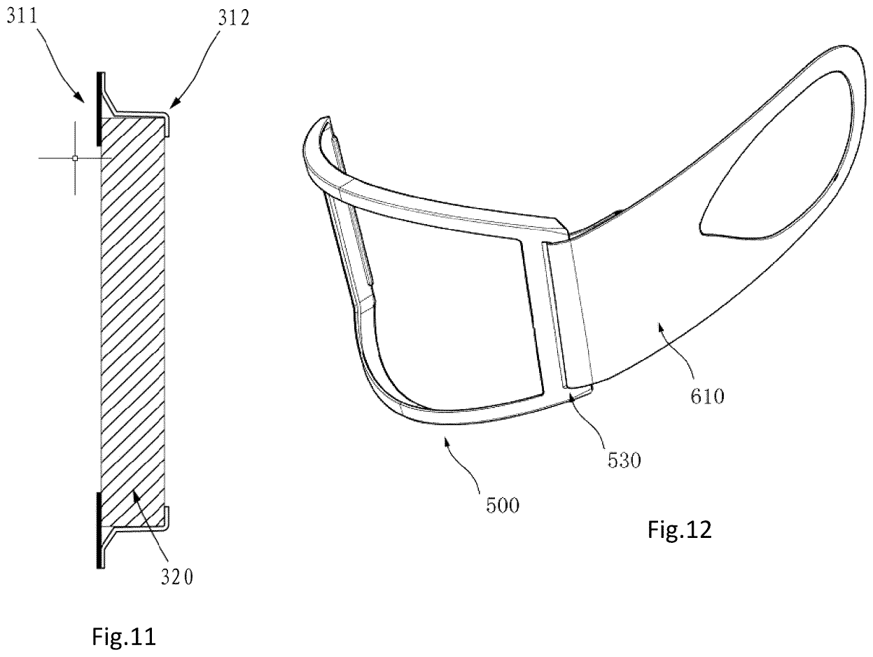

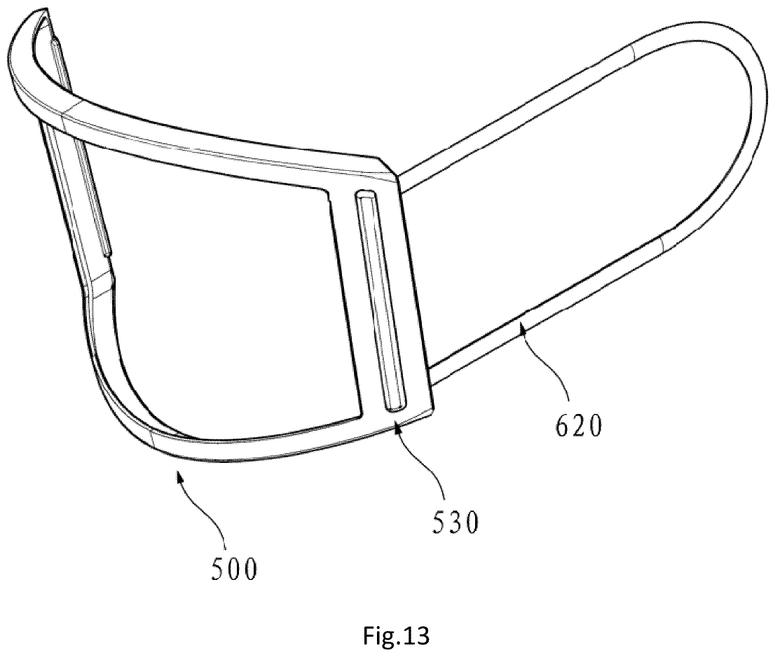

In some embodiments, referring to FIG. 1 to FIG. 13, the second frame 500 comprises an through hole 530 at a position close to the left side edge and a position close to the right side edge. The suspension assembly 600 comprises a belt 610. The belt 610 passes through the through hole 530 for connecting the second frame 500. The suspension assembly 600 may comprise a rope 620. The rope 620 passes through the through hole 530 for connecting the second frame body 500. In this embodiment, the belt 610 or the rope 620 is affixed through the through hole 530. As shown in FIG. 12, when the belt 610 is in use, one end of the belt 610 passes through the through hole 530 and is connected back to the belt 610. The other end of the belt 610 has a lug hole for hanging on an ear of the user. As shown in FIG. 13, when the rope 620 is in use, two ends of the rope 620 pass through the through hole 530 respectively and are affixed to the through hole 530. The rope 620 forms a lug hole for hanging on an ear of the user.

Preferably, when the belt 610 is in use, the through hole 530 has an elongated profile. The belt 610 does not need to be looped as a whole and passes through the through hole 530. Preferably, when the rope body 620 is in use, the mask comprises two through holes 530. The two ends of the rope 620 pass through the through holes 530 respectively and join together at the front side of the second frame body 500, so as to prevent the rope 620 from falling off from the through holes 530.

When the belt 610 is in use, other functional components may also be printed or attached to the belt 610, so as to improve the overall aesthetic and increase functionalities of the mask.

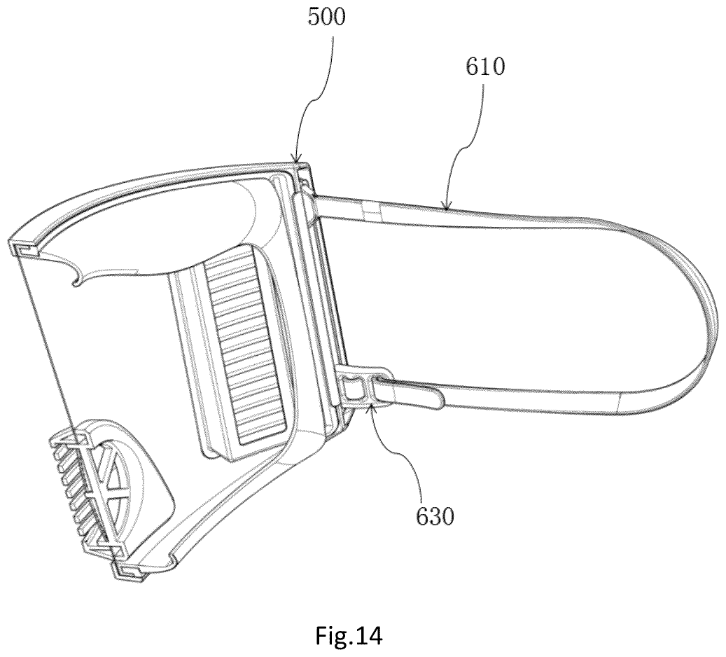

In some embodiments, referring to FIG. 14, the suspension assembly 600 comprises a belt 610 connected to the second frame 500. The belt 610 may have an adjustable length. The belt 610 may have its length adjusted with a hook-and-loop fastener, an adjustable buckle, or the like. As such. the length of the belt 610 can be adjusted according to the circumference of the head of different user making it comfortable for the user to wear. Meanwhile, the belt 610 can be adjusted to fit users with different head circumference. It has a wide range of applications and is convenient to use.

In some embodiments, the second frame 500 comprises openings 530 at a position close to the left edge and a position close to the right edge of the mask. The hanging assembly 600 further comprises an adjusting buckle 630. One end of the belt 610 is adapted to pass through one of the openings 530 for connecting to the second frame 500. The adjusting buckle 630 is attached at the other end of the belt 610. The other end of the belt 610 passes through the through hole 530 and is tied on the adjusting buckle 630. The length of the belt 610 can be adjusted by adjusting the position of the adjusting buckle 630 along the belt 610. If the position of the adjusting buckle 630 along the belt 610 is changed, the reserved length of the belt 610 is also changed, thereby achieving the purpose of adjusting the use length of the belt 610. Alternatively, the adjusting buckle 630 can be affixed on the second frame 500, and the other end of the belt 610 is looped and inserted on the adjusting buckle 630.

In some embodiments, referring to FIG. 1 to FIG. 13, a surface of the light-transmitting sheet 200 has an anti-haze cover layer disposed thereon, so as to extend the service life of the light-transmitting sheet 200. Optionally, the cover layer is also antibacterial. Optionally, the cover layer may be disposed on the surface of the light-transmitting sheet 200 as a film pasting or coating. The cover layer may be directly coated with an anti-haze and antibacterial plastic film.

In some embodiments, referring to FIG. 1 to FIG. 13, the mask comprises a plurality of filter assemblies 300. The bases 310 of the plurality of filter assemblies 300 are adapted to be installed at intervals on the inner side of the light-transmitting sheet 200 to improve the airflow, so as to maintain smooth breathing for the user.

Optionally, the filter assembly 300 may be disposed on a left side and a right side of the light-transmitting sheet 200 or around the periphery of the light-transmitting sheet 200.

In some embodiments, referring to FIG. 1 to FIG. 13, the filter element 320 comprises a filter sponge or a filter sheet having an accordion-folded structure. The filter element 320 may be adjusted according to the airflow required to be filtered. The entire filter material can even be used as a filter element 320.

Preferably, functional components such as an exhaust valve, a water absorption sponge or a sound amplification module may also be installed on the light-transmitting sheet 200.

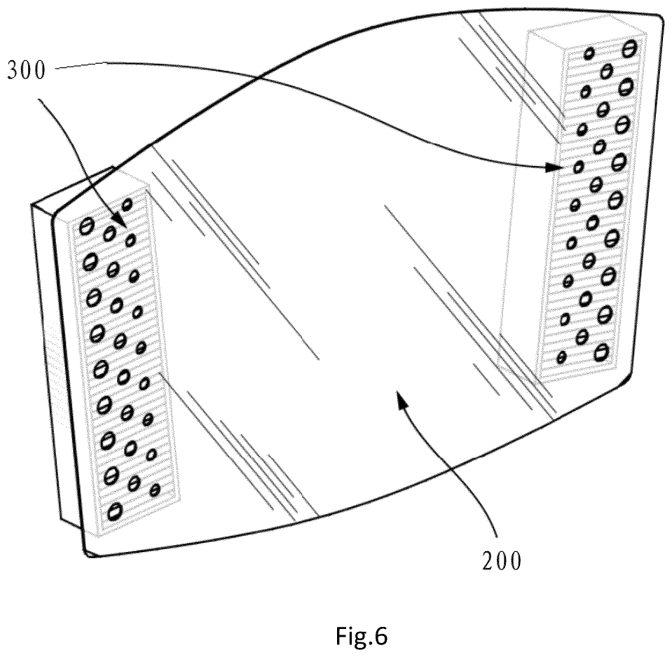

In some embodiments, referring to FIG. 6 to FIG. 8, the base 310 has a rectangular shaped frame. In another embodiment, as shown in FIG. 9 to FIG. 11, the base 310 comprises a plate 311 attached to the inner side of the light-transmitting sheet 200, and a cover 312 for covering the inner side of the plate 311. The ventilation channel is formed with the plate 311 and the cover 312. The rear side of the cover 312 has an opening communicating with the interior of the ventilation channel.

As shown in FIG. 6 to FIG. 8, there are a plurality of air holes 210. The plurality of air holes 210 are distributed in a scattered manner. As shown in FIG. 9 to FIG. 11, the air holes 210 are rectangular and are in communication with the opening of the ventilation channel.

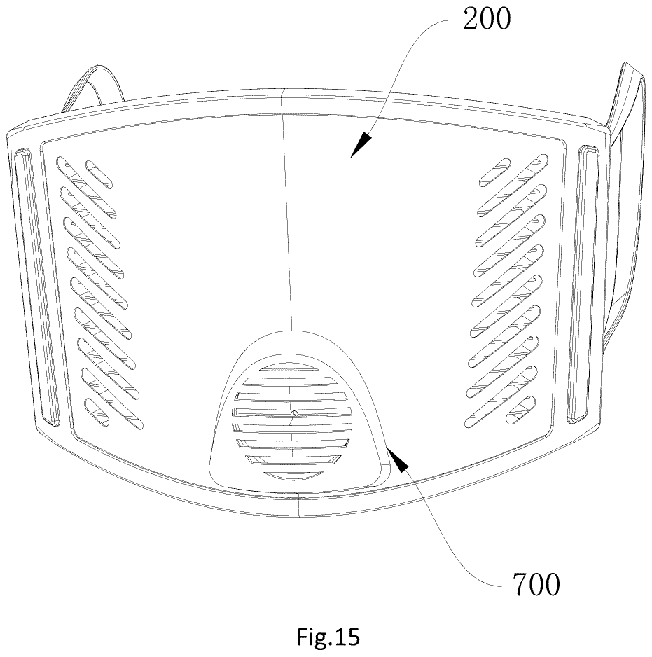

In some embodiments, referring to FIG. 14 and FIG. 15, the light-transmitting sheet 200 comprises an air outlet 220. The air outlet 220 has a one-way valve 700. The one-way valve 700 is adapted to guide the gas inside the light-transmitting sheet 200 to exit to the outer side of the light-transmitting sheet 200 and to prevent the gas outside the light-transmitting sheet 200 from entering to the inner side of the light-transmitting sheet 200. In this way, when the user inhales, air enters the ventilation channel through the air holes 210 on the light-transmitting sheet 200, and enters the enclosing space between the first frame 100, the light-transmitting sheet 200 and the user's face after being filtered by the filter element 320 in the ventilation channel. When the user exhales, the gas exhaled by the user enters the enclosing space. A part of the gas passes through the ventilation channel. After being filtered by the filter element 320 in the ventilation channel, a part of the exhaled gas exits from the air holes 210 on the light-transmitting sheet 200. The other part of the exhaled gas may be directly discharged through the one-way valve 700 without being filtered, so as to improve the exhale efficiency. This design is suitable for a user to perform high-intensity movement in an outdoor environment away from the crowd, so as to accelerate the exhaling and prevent the user from laboured breathing.

In some embodiments, referring to FIG. 14, the one-way valve 700 is adapted to be mounted on the light-transmitting sheet 200 to a position away from the lips of the user. In other words, the air outlet 220 is located on the light-transmitting sheet 200 and away from the position of the lips of the user. This will prevent the one-way valve 700 from blocking the lips. It may be understood that, in other embodiments of this application, the one-way valve 700 may not need to give way to the lip of the user. In this instance, the material of the one-way valve 700 may made from a light-transmitting material. As such, the position of the one-way valve is not a limiting factor.

Optionally, the one-way valve 700 is adapted to be mounted on the light-transmitting sheet 200. The one-way valve 700 is located below the nostrils of the user and away from the position of the lips. As such, the gas exhaled by the user can be discharged through the one-way valve 700 through a short distance, thereby improving the efficiency of gas discharge. It may be understood that, in other embodiments of this application, the one-way valve 700 may also be mounted on the light-transmitting sheet 200 at a position above the nostrils, the left side or the right side of the user. The position of the one-way valve is not a limiting factor here.

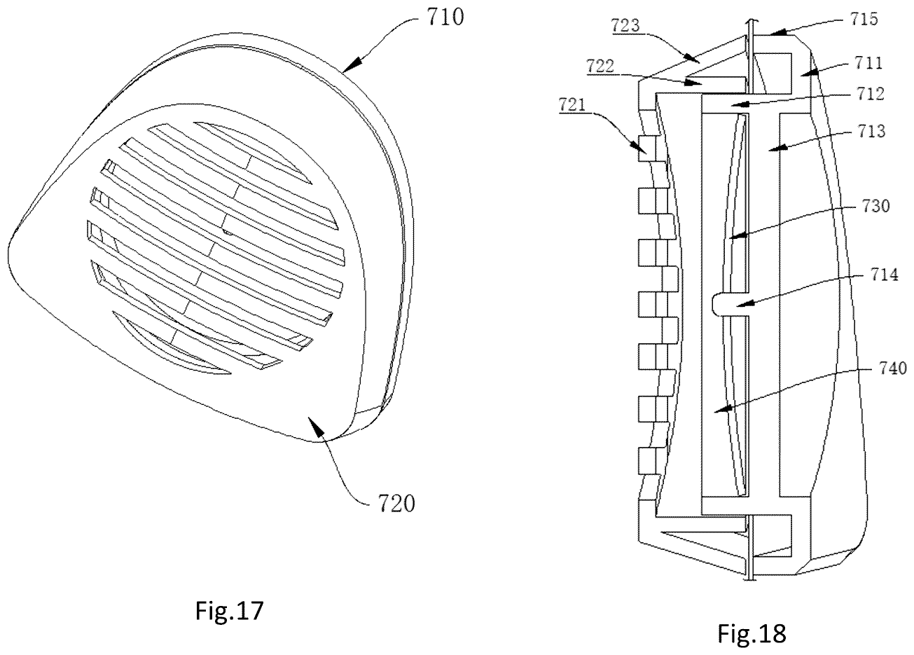

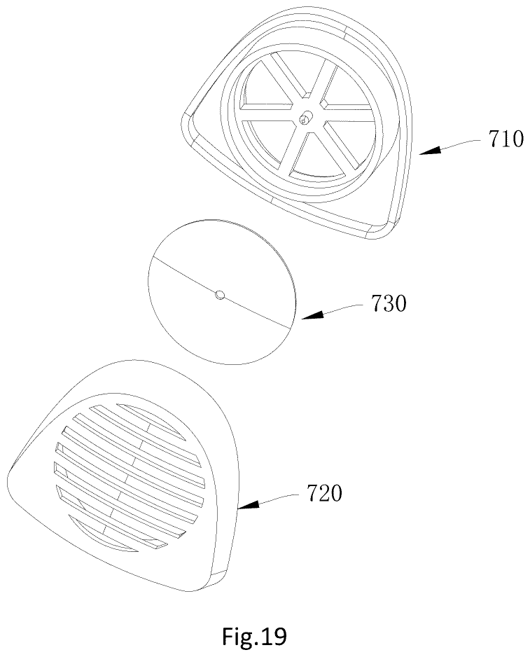

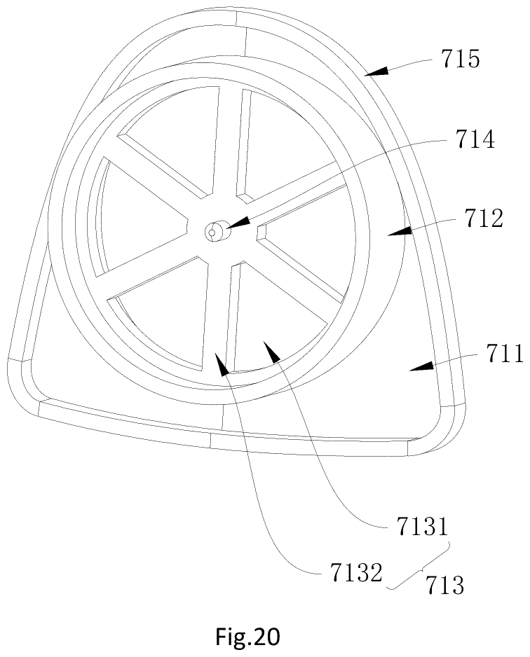

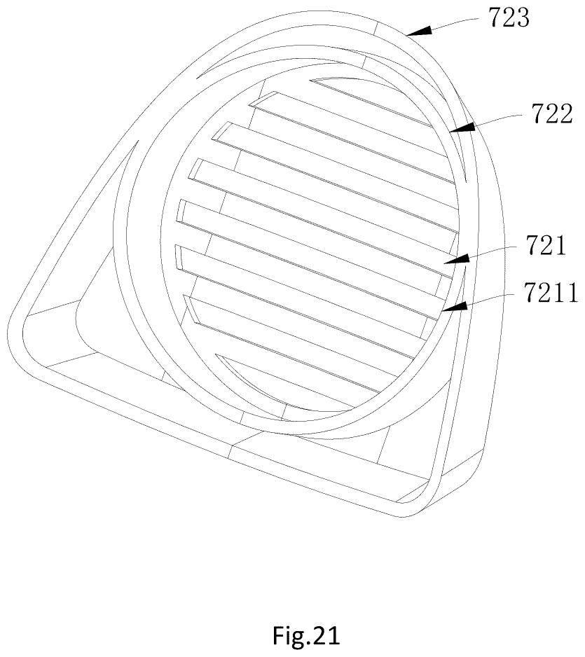

In some embodiments, referring to FIG. 17 to FIG. 20, the one-way valve 700 comprises a valve cavity 740. A mounting column 714 is secured inside the valve cavity 740. The mounting column 714 is sleeved with a diaphragm 730. The diaphragm 730 is concavely arranged to complement the inner side of the light-transmitting sheet 200. A peripheral edge of the diaphragm 730 can be squeezed out of a peripheral inner wall of the valve cavity 740 by the gas inside the diaphragm 730 for connecting an inner side of the light-transmitting sheet with an outside of the light-transmitting sheet 200. A peripheral edge of the diaphragm 730 can be pressed against a peripheral inner wall of the valve cavity 740 by a gas outside the diaphragm 730, thereby preventing gas outside the light-transmitting sheet 200 from entering into the inner side of the light-transmitting sheet 200. In this embodiment, since the diaphragm 730 is concave inwards relative to the inner side of the light-transmitting sheet 200, when the gas exhaled by the user acts on the diaphragm 730, the gas can push a peripheral edge of the diaphragm 730 to bend outwards and deform, so that a peripheral edge of the diaphragm 730 is separated from the inner wall of the valve cavity 740, and the gas can flow to the outer side between the diaphragm 730 and the inner wall of the valve cavity 740. When the user exhales, the gas acts on the outer side of the diaphragm 730 and pushes the diaphragm 730 to continuously bend inwards such that the peripheral edge of the diaphragm 730 can be more tightly attached to a circumferential inner wall of the valve cavity 740, and thereby preventing the gas outside the light-transmitting sheet 200 from flowing to the inner side of the light-transmitting sheet 200.

In some embodiments, referring to FIG. 17 to FIG. 20, the one-way valve 700 includes a base 710 and an outer cover 720. The base 710 and the outer cover 720 are detachably connected to the air outlet 220. The light-transmitting piece 200 is clamped between the base 710 and the outer cover 720. The base 710 and the outer cover 720 form an enclosed valve cavity 740. There are mounting column 714 disposed on the base 710 or the outer cover 720. A first through groove 7131 is formed on the base 710 in communication with the valve cavity 740. A second through groove 7211 is formed on the outer cover 720 in communication with the valve cavity 740. The base 710 and the outer cover 720 are detachably connected at the air outlet 220, so that the one-way valve 700 can be conveniently attached to and removed from the light-transmitting sheet 200. The parts are easy to maintain, clean, or replace, thereby prolonging the service life of the whole mask.

In some embodiments, referring to FIG. 17 to FIG. 20, the base 710 comprises a bottom plate 711 and a first convex ring 712 formed on the bottom plate 711. A peripheral edge of the bottom plate 711 engages an inner side of the light-transmitting sheet 200. The first convex ring 712 passes through the air outlet 220. A mounting plate 713 is formed on an inner wall of the first convex ring 712. The mounting column 714 is formed at the centre of the mounting plate 713. A peripheral edge of the diaphragm 730 engages a circumferential inner wall of the first convex ring 712. The outer cover 720 comprises a cover plate 721 and a second convex ring 722 formed on the cover plate 721. The second convex ring 722 is sleeved outside the first convex ring 712. During installation, the centre of the diaphragm 730 is securely sleeved upon the mounting column 714 first. Then. the first convex ring 712 of the base 710 is inserted into the air outlet 220, so that the bottom plate 711 engages the inner side of the light-transmitting sheet 200 while the first convex ring 712 engages the inner wall of the air outlet 220, Then, the second convex ring 722 of the outer cover 720 is sleeved outside the first convex ring 712. Specifically, the first convex ring 712 interference fits with the second convex ring 722, so that the base 710 and the outer cover 720 are firmly connected while detachable. In addition, the second convex ring 722 is required to engage the outer side of the light-transmitting sheet 200, so as to ensure that the second convex ring 722 is installed properly.

In some embodiments, referring to FIG. 17 to FIG. 20, a peripheral edge of the bottom plate 711 extends outwardly to form a first convex rib 715. A peripheral edge of the cover plate 721 extends outwardly to form a second convex rib 723. The first convex rib 715 and the second convex rib 723 engage an inner side and an outer side of the light-transmitting sheet 200 respectively. The first convex rib 715 and the second convex rib 723 are arranged in corresponding positions inside and outside of the light-transmitting sheet 200. The first convex rib 715 and the second convex rib 723 can be respectively clamped on the inner side and the outer side of the light-transmitting sheet 200 to prevent the base 710 or the outer cover 720 from being separated from the outer side or the inner side of the light-transmitting sheet 200 respectively. Since the light-transmitting sheet 200 has a thin sheet-shaped structure, the structural strength thereof is relatively low. The first convex rib 715 and the second convex rib 723 engage corresponding positions on the inner side and the outer side of the light-transmitting sheet 200, such that the first convex rib 715 and the second convex rib 723 have a shearing effect on the light-transmitting sheet 200 due to a staggered position.

Optionally, the first convex rib 715, the second convex rib 723 and the positions of the second convex ring 722 where the light-transmitting sheet 200 is contacted are designed to complement the radian of the light-transmitting sheet 200. This can improve the connections of the first convex rib 715, the second convex rib 723, the second convex ring 722, and the light-transmitting sheet 200.

Optionally, the mounting plate 713, the first convex ring 712, the second convex ring 722, and the cover plate 721 jointly form a valve cavity 740. The mounting plate 713 is installed opposite to the cover plate 721. The mounting column 714 extends outwardly from the centre of the mounting plate 713 to the direction of the cover plate 721.

Optionally, the mounting plate 713 has a plurality of first through grooves 7131 thereon. Each of the first through grooves 7131 is in communication with the valve cavity 740. Optionally, the mounting plate 713 includes a plurality of connecting strips 7132 distributed in a radial shape. A first through groove 7131 is formed between two adjacent connecting strips 7132. It can be understood that in other embodiments of this application, the first through groove 7131 may also be circular, elliptical, rectangular, or other polygons in shape.

Optionally, a plurality of second through grooves 7211 are formed on the cover plate 721. Each second through groove 7211 is in communication with the valve cavity 740. Optionally, the second through groove 7211 can be rectangular, circular, elliptical, or other polygonal.

The present disclosure relates to a powered respirator device that provides filtered breathable gases like air. The powered respirator device is a personal powered respirator device. The filtered breathable gases are filtered to a high level of filtration, and the respirator device further provides gases to maintain a continuous positive pressure to improve user comfort and augment breathing. The powered respirator device functions as a personal respiratory protection device designed to provide superior protection and breathing comfort. The respirator device functions as a lightweight, miniaturized Powered Air-Purifying Respirator (PAPR) system.

The powered respirator device 100 is a wearable unit containing a fan and a battery, which actively draws ambient air through a high-efficiency particulate air (HEPA) filter, rated at N95 or equivalent. This filtered air is then supplied to a comfortable, low-profile facepiece, creating positive pressure that reduces breathing resistance and prevents CO2 buildup. Key technical features include an active air supply mechanism, replaceable N95-rated HEPA filters, a lightweight and ergonomic design with the fan/filter unit situated away from the face for better heat dissipation and user comfort, and a disposable or easily cleanable facepiece. The system aims to offer continuous, effortless breathing with filtered air, akin to being maskless but with protection.

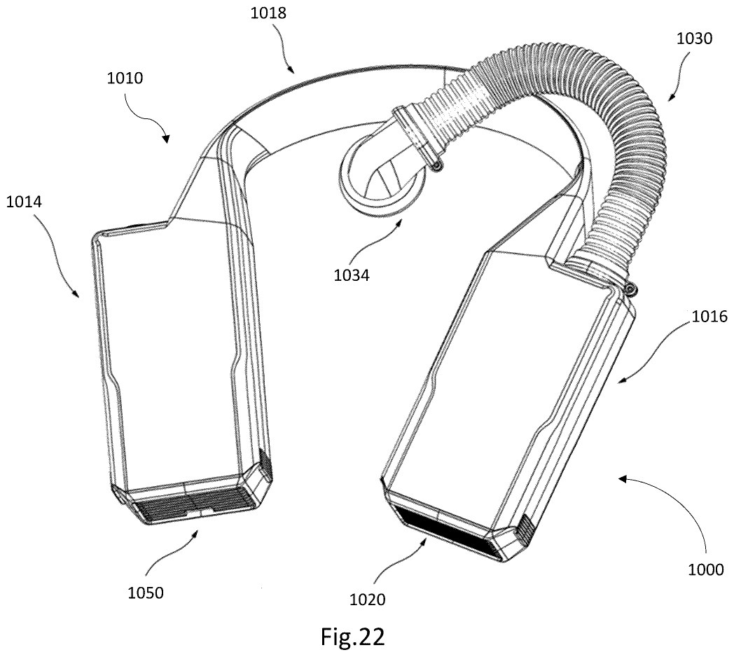

FIG. 22 illustrates an example embodiment a powered respirator device 1000 comprising a wearable air supply unit 1010, the wearable air supply unit 1010 comprising a blower 1012, a controller 1060 and a filter 1020. In use, the wearable air supply unit 1010 may be adapted to be worn around a user's neck or on a user's shoulders. The respirator device 1000 comprises a conduit 1030 fluidly coupling the wearable air supply unit 1010 to a mask 1040. The blower 1012 is configured to draw ambient air through the filter 1020 and deliver a continuous flow of filtered air to the mask 1040 via the conduit 1030 to maintain a continuous positive pressure within the mask 1040.

The wearable air supply unit 1010 comprises a first housing 1014 and a second housing 1016. The first and second housings are spaced apart from each other and connected by a bridging member 1018. The bridging member or connecting member 1018 extends between the first housing 1014 and the second housing 1016. The bridging member 1018 is adapted to be suspended from a user's body to support the wearable air supply unit 1010. The wearable air supply unit 1010 is adapted to be worn around the neck and/or shoulders of a user.

The bridging member 1018 may be a neck band adapted to be worn around a posterior aspect of a user's neck. The first housing 1014 and second housing 1016 are configured to rest on a user's chest and/or shoulders to distribute wearable air supply unit's weight across the chest and/or shoulders and/or upper back.

The neck band 1018 comprises a substantially U shape or an arcuate shape. The neck band 1018 may be any other arcuate shape that may be able to be placed over the shoulders or over the clavicles of the user. The first housing 1014 and second housing 1016 may be identical in size and shape such that the weight of the wearable air supply unit is evenly distributed over the user's upper body. The neck band 1018 may be made from a malleable material or a pliable material such that the shape can be changed and customized by each user. The neck band 1018 may include a rigid core embedded within a soft cushion material for user comfort. The first and second housings are positioned forward (proximate to the chin/clavicle), which serves the dual purpose of weight distribution and minimizing the length of the air conduit to reduce pneumatic drag. This reduces accidental dislodgement of the mask 1040 and more effectively distributes weight of the air supply unit 1010 over a user's shoulders, chest and upper back.

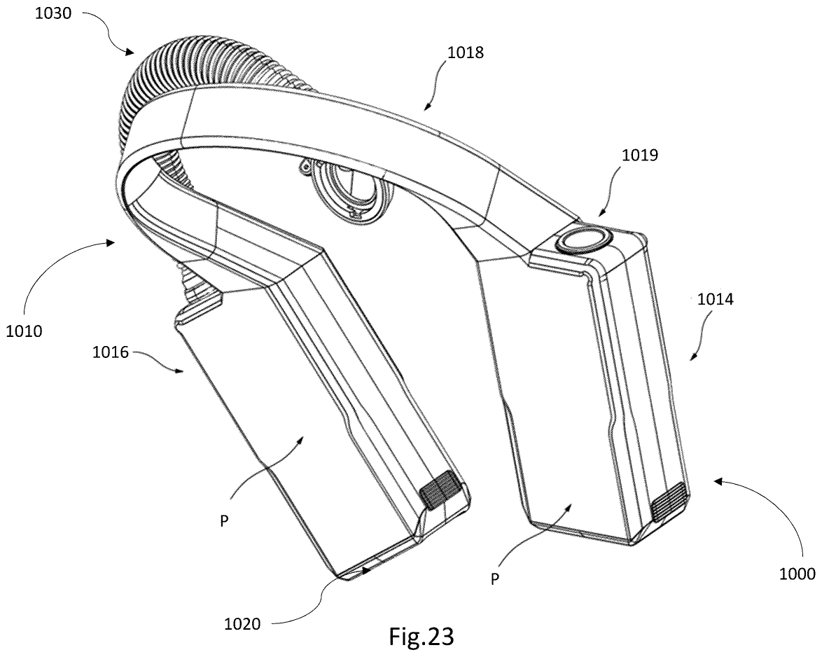

As shown in FIG. 23 the rear of each housing may include a soft padding to provide comfort for the user. Each housing, in use, may rest against the chest and/or clavicle of the user. The soft padding P on the rear face i.e., user proximal face provides padding for the user and improves comfort. The respirator device 1000 may include an ON/OFF button 1019 positioned on the first housing 1014, as shown in FIG. 23. The ON/OFF button may activate or deactivate the blower 1012.

The wearable air supply unit contains a power supply 1050. The power supply may be a battery. The battery may comprise multiple cells. The battery 1050 may be a rechargeable battery. The second housing 1016 contains the blower 1012 and the filter 1020. The filter 1020 may be a replaceable filter. The filter 1020 may be removable from the second housing 1016. The power supply 1050 is electrically coupled to the blower 1012 to provide power to the blower 1012. The replaceable filter 1020 may be removable from the second housing 1016. The replaceable filter 1020 may be a HEPA (High Efficiency Particulate Air) filter. Alternatively, the filter 1020 may be a high-grade MERV filter (e.g., a MERV 13-16 filter), or a ULPA filter.

The filter 1020 is preferably rated to at least a N95 level of filtration. The filter 1020 is positioned upstream of the blower 1012 in the second housing. The blower 1012 is adapted to draw ambient air through the filter 1020 to filter air before the air is delivered to the mask.

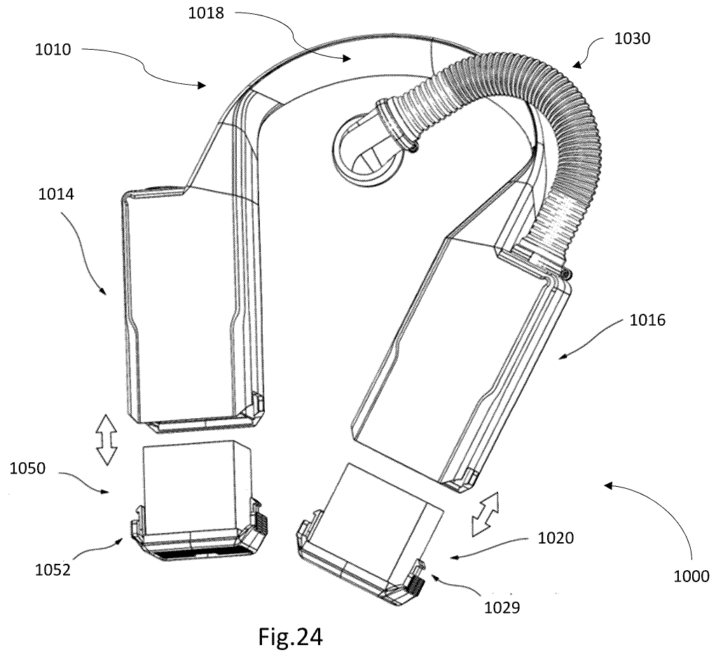

The powered respirator device 1000 may comprise one or more consumables. The one or more consumables may be removable and replaceable components. The consumables may be replaced with new parts. FIG. 24 illustrates the removable and replaceable consumables. The removable battery 1050 may be a consumable that can be replaced. The filter 1020 may be removable and replaceable filter. Each consumable component may include clips that allow removable attachment to wearable air supply unit.

As shown in FIG. 24, the removable battery 1050 comprises battery clips 1052. The battery clips may be releasable clips. The battery clips 1052 may be finger actuatable clips. In use, the battery clips 1052 may be squeezable by a user to release the battery 1050 from the first housing 1014. The battery 1050 is adapted to slide out of the first housing 1014.

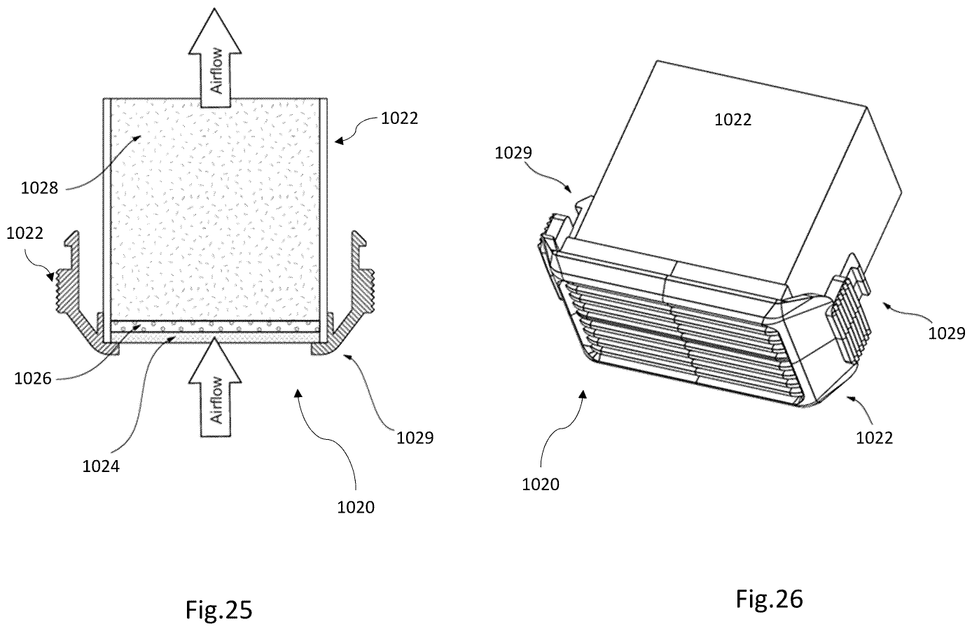

The filter 1020 (i.e. filter module) may be a releasable filter assembly i.e., a releasable filter module. The filter 1020 may be releasably connected to the second housing 1016. FIGS. 25 and 26 illustrate a detailed views of the filter 1020 (i.e., filter module). Referring to FIG. 25, the replaceable filter 1020 comprises a filter housing 1022, a protection mesh 1024, a pre-filter 1026 and a main filter 1028 (i.e. main filter medium). The pre-filter 1026 is preferably positioned between the protection mesh 1024 and the main filter 1028. The pre-filter 1026 may be positioned upstream of the main filter 1028. The pre-filter 1026 may comprise a coarse filter medium and the main filter 1028 may comprise a finer filter medium. The main filter 1028 may be a pleated HEPA filter. At least the main filter 1028 may be a HEPA filter that is rated to an N95 level of filtration.

Referring to FIG. 25 the protection mesh 1024 may be installed in the housing 1022. The mesh 1024 may protect the main filter 1028 from large particulate matter. The protection mesh 1028 may also protect from foreign objects entering the filter and entering the blower and damaging it. The protection mesh 1024 may include openings to allow ambient air to be drawn into the housing 1016 by the blower. Preferably the protection mesh may be adhered to or installed into or welded into the filter housing 1022. The protection mesh 1024 may be made from metal such as, for example, stainless steel or aluminum.

As shown in FIG. 26 the filter housing 1022 comprises a pair of actuatable clips (i.e., filter clips) 1029 adapted to removably fasten the filter 1020 to the second housing 1016. The filter clips 1029 may include two finger actuatable members that may resiliently moveable. The actuation members may be pushed toward each other to unfasten the filter 1020 from the second housing 1016. Alternatively, the clips 1029 may include finger actuatable members that may slide axially or may rotate to unfasten the filter 1020 from the second housing 1016. The filter 1020 (i.e. filter module) is adapted to slide out of the base of the second housing, as shown in FIG. 26, and the filter 1020 is held in an operative position via the filter clips 1029.

The flexible conduit 1030 may be made a corrugated tube as shown in FIGS. 22, 24 and 27. The conduit 1030 may include a spirally wound bead to provide rigidity to the conduit. The flexible conduit 1030 may be made from a medical grade plastics material. The flexible conduit 1030 may be at least 0.5 m long. The flexible conduit 1030 may be a flexible, lightweight hose that fluidly connects the air outlet of the wearable air supply unit to the inlet port on the facepiece. This conduit 1030 is designed to allow freedom of movement for the user.

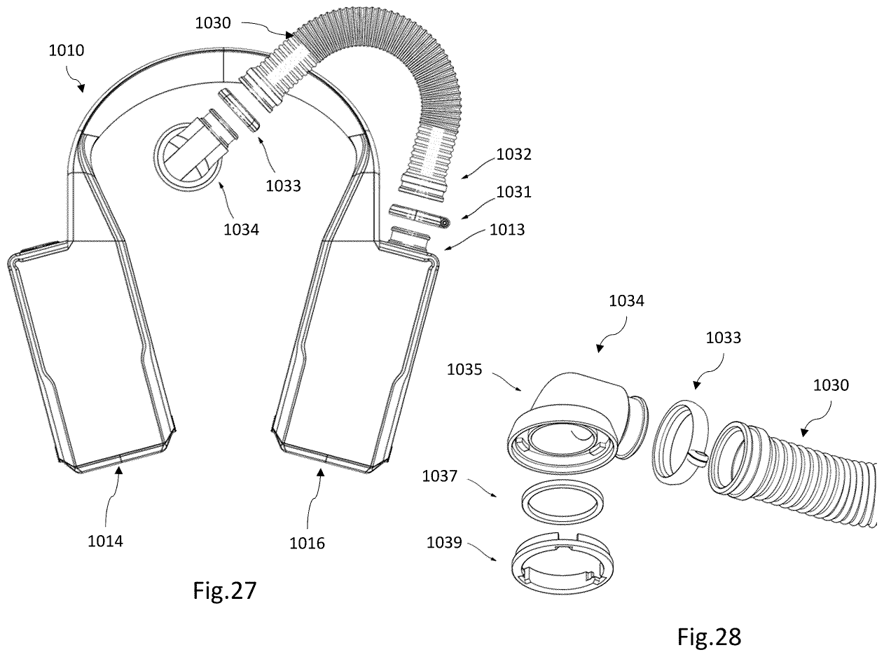

The conduit comprises a unit end connector 1032 to connect to the wearable air supply unit 1020 and a mask end connector 1034 adapted to connect to the mask 1040. FIG. 27 illustrates an example of the flexible conduit and its parts. FIG. 28 illustrates details of the mask end connector of the flexible conduit. Referring to FIG. 27, the flexible conduit 1030 comprises two collars 1031, 1033. Each collar is positioned at an end of the conduit 1030. A first collar 1031 is positioned between the unit end connector 1032 and an outlet 1013 of the wearable air supply unit 1010. A second collar 1033 is located between a proximal end of the conduit and the mask end connector 1034. The first collar 1031 couples the unit end connector 1032 to the outlet of the wearable air supply unit. The second collar 1033 couples the proximal end of the conduit 1030 to the mask end connector 1034. The collars 1031, 1033 may provide a sealing connection.

FIG. 28 illustrates details of the mask end connector 1034. Referring to FIG. 28, the mask end connector 1034 comprises a connector housing 1035, the connector housing defining a gas outlet opening 1036, a sealing ring 1037 and a locking ring 1039. The sealing ring 1037 is positioned around the gas outlet opening 1036 and a locking ring 1039 attached to the connector housing 1035. The locking ring 1039 is configured to releasably engage with a corresponding connector on the mask. The locking ring 1039 may lock with a connector on the mask e.g., a mask inlet by a push fit or a friction fit. Alternatively, the locking ring 1039 may engage with a mask connector e.g., a mask inlet by a bayonet fitting or a twist action to engage the locking ring with a corresponding connector on the mask inlet.

The mask end connector 1034 may define a quick connect docking system that allows a user to quickly connect and disconnect the mask end connector 1034 from the mask 1040. The mask end connector 1034 may allow a user to connect and disconnect the conduit from the mask by a single movement i.e., a single action. The quick connect docking system may include a release button or actuator that may be located on the interior rim of the mask to prevent accidental disconnection and to maintain a low-profile exterior.

The mask end connector 1034 may include an elbow formed therein as shown in FIG. 28. The elbow may be a pre-formed curvature adapted to reduce kinking and accommodate variations in user neck-to-face dimensions, and to orient the proximal end of the conduit such that it connects to the mask with the conduit 1030 running substantially parallel to a plane of the user's face for unobtrusiveness. The unit end connector 1032 may be planar i.e. substantially straight. Alternatively, the unit end connector may comprise an elbow to direct the conduit away from the wearable air supply unit 1010

The mask 1040 may comprise a filtered mask inlet port, an exhalation port, and an inlet connector i.e., a tube docking connector integrated into the filtered mask inlet port to allow quick connection and disconnection of the mask end connector of the conduit 1030. The tube docking connector may comprise one or more engagement protrusions that are configured to engage with one or more engagement slots in the locking ring 1039 of the mask end connector 1034 of the conduit to form a connection between the mask and the conduit.

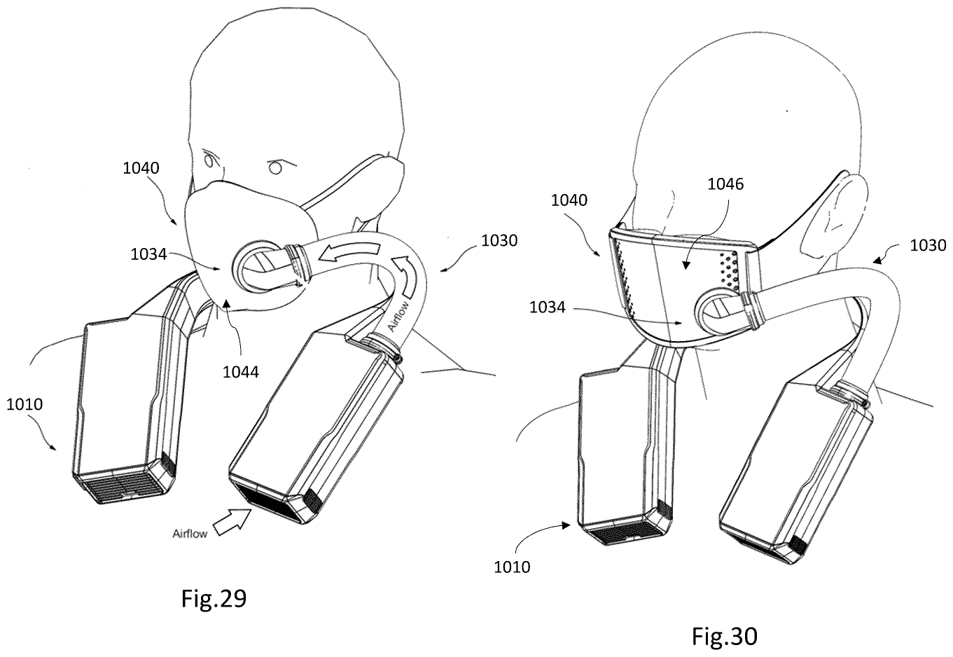

FIGS. 29 to 30 illustrate examples of different face masks that may be with the powered respirator device 1000. The mask 1040 may be a separate element from the respirator device 1000. Alternatively, the mask 1040 may be part of the respirator device 1000. The mask 1040 may be consumable component of the powered respirator device 1000. The mask 1040 may be adapted to connect to the conduit 1030 to receive filtered air from the wearable air supply unit 1010. The mask 1040 may be a full face 1042 i.e., a total face mask, a disposable fabric face mask 1044 or a reusable elastomeric face mask 1046.

FIG. 29 illustrates an example of a disposable face mask 1046 being used by the user. The disposable face mask 1046 is adapted to cover at least the nose and mouth of the user. The disposable face mask 1046 may be made from a fabric material. The disposable face mask 1046 is adapted to connect to the conduit 1030 via the mask end connector 1034. The disposable face mask 1046 is adapted to receive filtered air from the wearable air supply 1010 through the conduit 1030.

FIG. 30 illustrates an example of a reusable elastomeric mask 1044. The reusable elastomeric face mask 1044 is adapted to cover at least the nose and mouth of the user. The reusable face mask 1044 includes one or more vents formed in a body of the mask to vent exhaled gases. The mask 1044 connects to the conduit 1030 and receives filtered air from the wearable air supply unit 1010. The reuseable elastomeric mask 1044 may seal around the nose and mouth of the user.

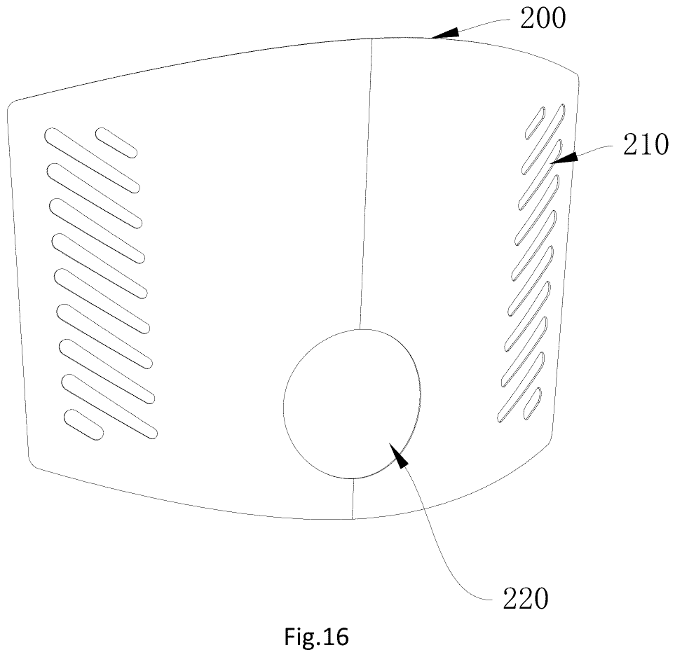

In one example, the mask 1040 may be a mask may be a mask as described in reference to FIGS. 1 to 21. The mask may include an inlet and the conduit 1030 may fluidly connect to the mask. The mask of FIGS. 1 to 21 may be configured to receive filtered air from the wearable air supply unit to the mask. For example, the mask comprises: a first frame for covering a user's face, wherein the first frame has a first opening opposite to the lips of the user, a light-transmitting sheet engaging an edge of the first opening to cover the first opening, such that light on an inner side of the first frame can be transmitted to an outer side of the first frame through the light-transmitting sheet, wherein the light-transmitting sheet is convex, the base is arranged on the inner side of the light-transmitting sheet and is a has a ventilation channel internally, the light-transmitting sheet comprises air holes in communication with the ventilation channel.

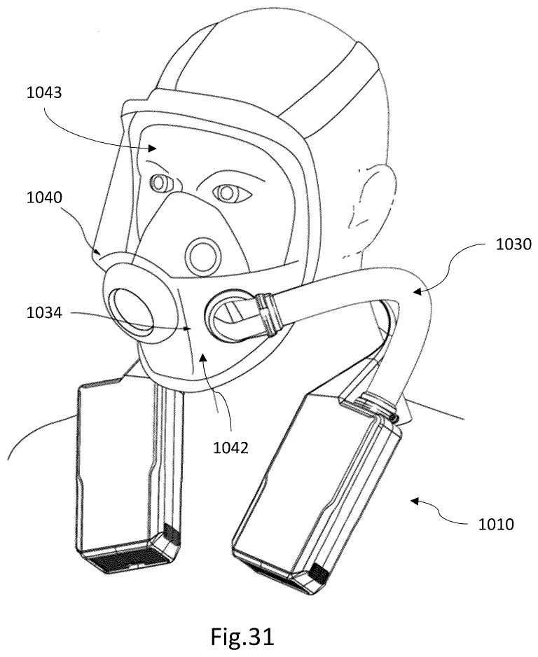

FIG. 31 illustrates an example of a full-face mask 1042 i.e. a total face mask. The total face mask 1042 covers the eyes, nose and mouth of the user. The full-face mask 1042 is configured to seal on a region of the face surrounding the eyes, nose and mouth. The full-face mask comprises a transparent visor 1043 to clear vision for the user.

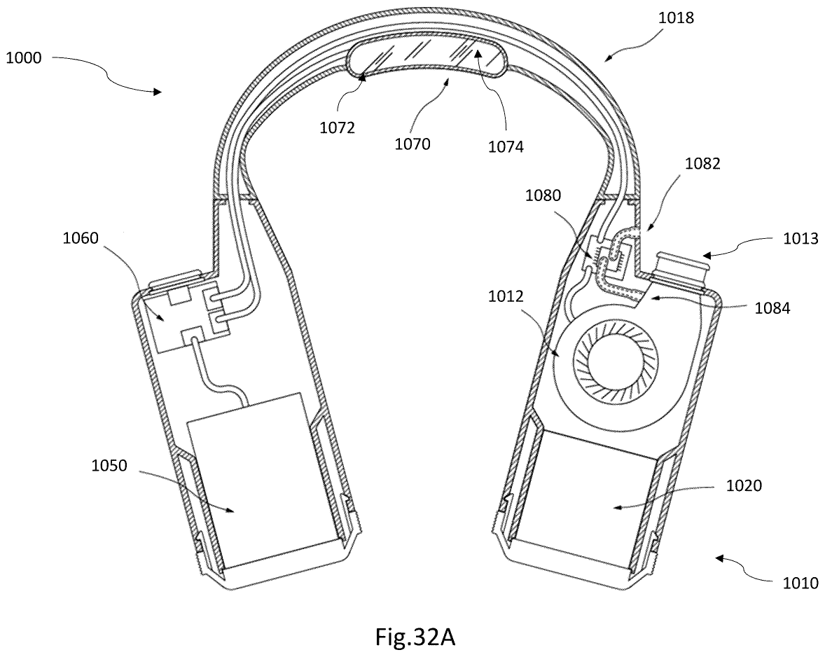

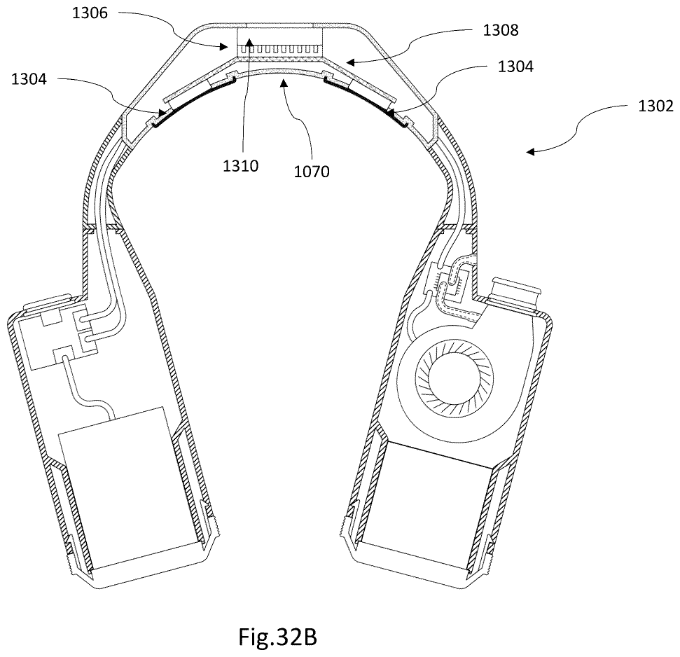

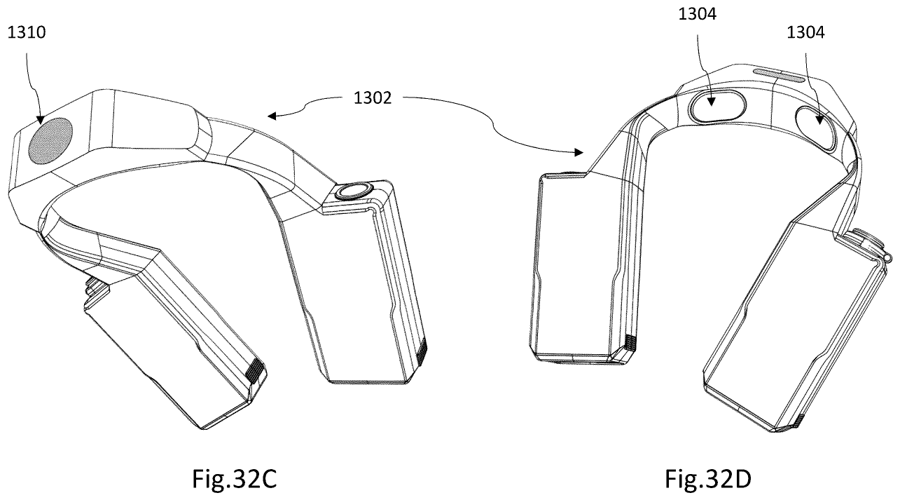

FIG. 32 illustrates the internal components of the wearable air supply unit 1010. Referring to FIG. 32, the wearable air supply unit 1010 comprises a thermoelectric cooling (TEC) device 1070 integrated into the bridging member 1018. The TEC device 1070 (i.e., TEC module) is configured to provide heat dissipation to a user and provide localized cooling. The TEC device 1070 may be integrated into the neck band 1018 such that the TEC device 1070 can contact a user's neck in use. Preferably the TEC device 1070 (i.e., TEC module) is located on an inner surface of the neck band 1018. The TEC device 1070 is preferably positioned to contact a posterior aspect of the user's neck in user. The TEC device 1070 comprises a thermoelectric substrate 1072 and a proximal surface 1074 adapted to be contact the user's skin in the posterior aspect of the neck. The TEC device 1070 comprises a distal surface exposed to ambient air. The TEC device 1070 is electrically coupled to the battery 1050. The TEC module 1070 is activated by power from the battery 1050. The substrate may be powered by power from the battery 1050 and configured to actively transfer heat from its proximal surface to its distal surface, thereby providing the localized cooling to the user's neck.

The TEC device 1070 may be integrated into the neck band 1018. Alternatively, the TEC device 1070 may be removably mounted into the neck band 1018. The TEC device 1070 (i.e., TEC module) may be a replaceable component. In an alternative configuration the powered respirator device 1000 may comprise a Peltier device or an Electrocaloric cooling device or other solid state electro-cooling device to provide localized cooling.

The powered respirator device 1000 comprises at least one sensor 1080 operatively coupled to the controller 1060 and adapted to transmit a detected parameter to the controller 1060. The sensor 1080 may be configured to detect at least one of an environmental parameter or a physiological parameter. Preferably, the controller is programmed to modulate a speed of the blower in real-time based on data received from the at least one sensor to optimize filtration effectiveness and/or optimize power consumption of the power supply 1050. The controller 1060 is configured to control the blower based on real-time data received from the at least one environmental or physiological sensor 1080, thereby optimizing at least one of filtration effectiveness, user comfort, or power consumption of the rechargeable power supply.

Preferably the sensor 1080 comprises a pressure sensor adapted to measure pressure of the flow of gases from the blower 1012. The pressure sensor 1080 may be placed on a circuit board. The pressure sensor 1080 may further include an ambient pressure port 1082 fluidly connected to ambient air and a blower outlet pressure port 1084 fluidly connected to the blower outlet, as shown in FIG. 32 and FIG. 34. The pressure sensor 1082 measures pressure by subtracting the pressure of blower outlet by the ambient pressure to determine pressure of the gases from the blower. The pressure sensor 1082 is configured to determine an inhalation phase or a breathing pattern of a user based on the measured pressure. Preferably the pressure sensor 1080 is configured to determine an inhalation phase of the user. The controller 1060 is further configured to control the blower to provide gases synchronized with the inhalation phase or breathing pattern of the user such that the flow is higher during an inhalation phase as compared to an exhalation phase.

In an alternative configuration the sensor comprises a particulate sensor adapted to detect the ambient particulate matter. The controller 1060 controller is configured to: increase blower speed in response to an increase in detected ambient particulate matter or control the blower 1012 to a blower speed that is correlated to the measured ambient particulate matter.

In an embodiment the powered respirator device comprises a wireless connectivity module e.g., a Bluetooth module or a Cellular module, configured to transmit system performance data and filter life status to a mobile device.

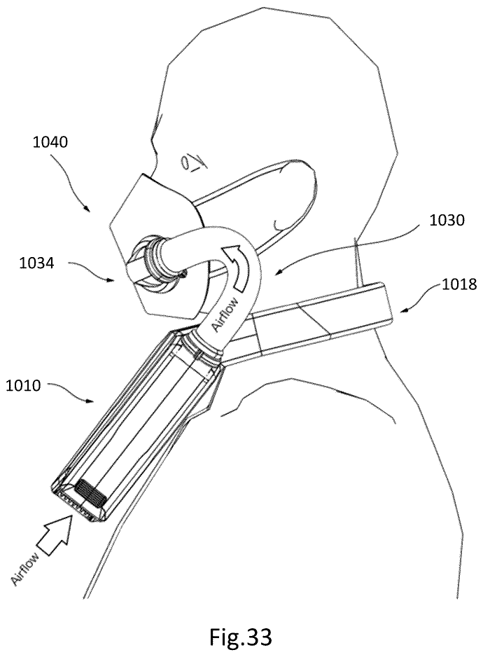

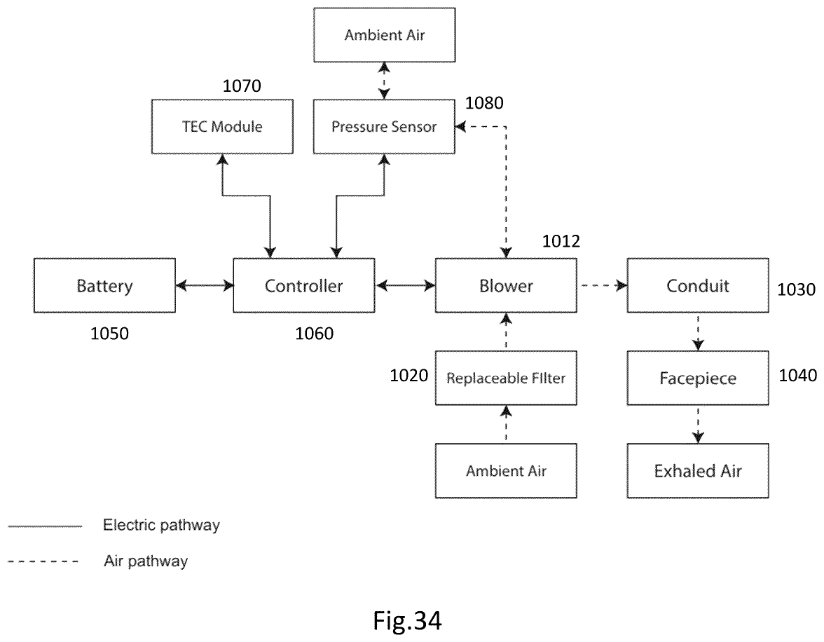

FIG. 33 illustrates an example of a method of operation and use of the powered respirator device 1000. Referring to FIG. 33, the user dons the wearable air supply unit 1010. The user places the neck band 1018 around the user's neck and the first and second housings rest on the user's chest. The user attaches the mask 1040 over the nose and mouth creating a seal around the nose and mouth. The user connects the conduit 1030 between the air supply unit 1010 and the mask 1040, such that the conduit defines a gas path between the air supply unit 1010 and the mask 1040. The user connects the battery 1050 and the filter 1020 (e.g., HEPA filter). The user activates the blower via a user interface e.g. a button or a touch screen. The blower draws in ambient air through the HEPA filter 1020. The filtered air is delivered to the mask for the user to inhale. The controller 1060 is configured to control the blower 1012 to provide a constant flow rate or provide a flow rate to create a constant positive pressure in the mask 1040. Exhaled air is expelled from the mask through an exhalation valve or is exhaled through another designated path. The filter 1020 (e.g., HEPA filter) and the mask 1040 may be replaced periodically based on the usage and environmental conditions to maintain performance and hygiene.

FIG. 34A illustrates an electric and pneumatic architecture of the powered respirator device 100. The solid lines indicate electrical connections. The dashed lines indicate pneumatic connections. Referring to FIG. 34A, the controller 1060 is electrically connected to the battery 1050 and receives power from the battery 1050. The controller 1060 is electrically coupled to the TEC device 1070 (i.e., TEC module) and adapted to activate an element in the substrate to create the thermoelectric effect and cause localized cooling. The controller 1060 is in communication with pressure sensor 1080 and is adapted to receive pressure values of the air from the blower 1012. The pressure sensor 1080 may measure ambient pressure and a pressure of the gases from the blower 1012. The controller 1060 may further be configured to determine a pressure signal of a user breathing by filtering the pressure from the blower and determining oscillations in the filtered pressure signal to determine user breathing e.g., user respiratory rate or user breathing phases. The controller 1060 is electrically coupled to the blower 1012 and configured to operate the blower 1012. The controller 1060 may control the blower 1012 to provide gases synchronized with the inhalation phase or breathing pattern of the user such that the flow is higher during an inhalation phase as compared to an exhalation phase. The controller 1060 controls the blower 1012 to provide a flow of air to the mask 1040 to create a positive pressure in the mask. The controller 1060 may further be configured to control the blower 1012 to provide a flow of gases such that the gases are filtered by the filter 1020. The blower 1012 is fluidly connected to the conduit 1030 and the replaceable filter 1020. The conduit 1030 is fluidly coupled to the mask 1040 (i.e., facepiece) and exhaled air is expelled from the mask 1040 to ambient. As shown in FIGS. 32B, 32C, and 32D, there is shown another alternative powered respirator device 1302 which includes a thermoelectric cooling feature (TEC) 1070. In this example embodiment, the TEC 1070 feature is placed at the neckband of the device 1302 and as shown in the cross section view of FIG. 32B, the TEC cooling modules 1304 are arranged to contact the back of a user's neck when the device 1302 is worn, and in turn offer a cooling effect to the user when the TEC cooling modules 1304 are activated. This is particularly advantageous in hot climates where cooling of a user's neck would assist to reduce the body temperature of the user. As shown, the TEC cooling modules 1304 may include a heat conductive plate, which is used to remove the heat from the cooling modules 1034. This heat conductive plate 1308 may be engaged with a heatsink 1306 and in turn may be cooled with an optional fan 1310 to expel the heat away from the user. Preferably, as shown in FIGS. 32B, 32C and 32D, the heat is expelled by a fan 1310 inside the TEC module 1070 and directly out of the TEC module away from the neck band (and user) so as avoid recirculating the heat back to the TEC module 1070 and reduce the operation efficiency of the TEC module 1070.

The respirator device 1000 comprises an active air supply mechanism, replaceable N95-rated HEPA filters, a lightweight and ergonomic design with the fan/filter unit situated away from the face for better heat dissipation and user comfort, and a disposable or easily cleanable facepiece. The respirator device 1000 offers continuous, effortless breathing with filtered air, akin to being maskless but with protection.

For individuals with chronic respiratory conditions (e.g., asthma, allergic rhinitis), the respirator device 1000 offers improved mobility and quality of life by providing comfortable, long-term protection against airborne triggers. In industrial and occupational settings (e.g., construction, manufacturing, healthcare), the respirator device 1000 can enhance worker productivity, safety, and comfort, especially in hot environments or where prolonged mask use is mandatory. This can lead to better compliance with safety standards. The powered respirator device 1000 as described herein, benefits these sectors by offering a reusable, cost-effective alternative to daily disposable N95 masks or other disposable masks.

The powered respirator device 1000 is advantageous because it actively supplies a continuous flow of filtered air to the mask (i.e. facepiece). This creates a positive pressure within the mask, reducing breathing resistance compared to passive N95 masks. This makes it easier for users, especially those with compromised lung function, to breathe.

The powered respirator device 1000 includes a HEPA filter. The HEPA filter is designed to remove at least 95% of airborne particulates, including dust, allergens, pollutants (like PM2.5), bacteria, and viruses. The respirator device 1000 provides a high degree of filtration and efficient filtration.

The powered respirator device 1000 provides enhanced comfort and thermoregulation. By situating the battery, fan and filter away from the face i.e., on the chest in the first and second housings. The respirator device 1000 helps to reduce heat and moisture buildup within the mask, providing a cooling effect. The respirator device 1000 is designed to be lightweight, comfortable for prolonged use, and less obtrusive than traditional industrial PAPRs, promoting better user compliance and mobility.

Reference throughout this specification to “one embodiment”, “some embodiments”, “an exemplary embodiment”, “an example”, “a specific example”, or “some examples”, means that a particular feature, structure, material, or characteristic described in connection with the embodiment or example is included in at least one embodiment or example of the present application. In this specification, the schematic representation of the above terms does not necessarily refer to the same embodiment or example. Furthermore, the particular features, structures, materials, or characteristics may be combined in any suitable manner in any one or more embodiments or examples.

The above are merely optional embodiments of the present application and are not intended to limit the present application. For those skilled in the art, the present disclosure may have various modifications and changes. Any modifications, equivalent replacements, improvements and the like made within the spirit and principle of the present application shall fall within the scope of the claims of the present application.

1. A powered respirator device comprising:
a wearable air supply unit, the wearable air supply unit comprising a blower, a controller and a filter, wherein the wearable air supply unit is adapted to worn around a user's neck or on a user's shoulders,
wherein the blower is configured to draw ambient air through the filter and deliver a continuous flow of filtered air to a mask worn by a user to maintain a continuous positive pressure within the mask.

2. The powered respirator device of claim 1, wherein the wearable air supply unit comprises a first housing and a second housing, the first and second housing being spaced apart from each other and connected by a bridging member, wherein the bridging member adapted to be suspended from a user's body to support the wearable air supply unit.

3. The powered respirator device of claim 2, wherein the bridging member is a neck band adapted to be worn around a posterior aspect of a user's neck and wherein the first housing and second housing are configured to rest on a user's chest and/or shoulders to distribute wearable air supply unit's weight across the chest and/or shoulders and/or upper back.

4. The powered respirator device of claim 2, wherein the first housing contains a power supply, wherein the power supply is a battery and the second housing contains the blower and the filter, wherein the filter is a replaceable filter, and the power supply is electrically coupled to the blower to provide power to the blower, and the second housing being spaced away from the mask.

5. The powered respirator device of claim 4, wherein the replaceable filter is a HEPA (High Efficiency Particulate Air) filter that is rated to at least a N95 level of filtration and the filter is upstream of the blower.

6. The powered respirator device of claim of claim 5, wherein the replaceable filter comprises a filter housing, a protection mesh, a pre-filter and a main filter, wherein the pre-filter is positioned between the protection mesh and the main filter, wherein the pre-filter is upstream of the main filter and the filter housing comprising a pair of actuatable clips adapted to removably clip to the second housing.

7. The powered respirator device of claim 6, wherein at least the main filter is rated to at least a N95 level of filtration.

8. The powered respirator device of claim 6, comprising a conduit fluidly connected to the wearable air supply unit, wherein the conduit is a flexible conduit, the conduit comprises a unit end connector to connect to the wearable air supply unit and a mask end connector adapted to connect to a mask, wherein the mask end connector comprises a connector housing, the connector housing defining a gas outlet opening, a sealing ring positioned around the gas outlet opening and a locking ring attached to the connector housing, wherein the locking ring is configured to releasably engage with a corresponding connector on the mask.

9. The powered respirator device of claim 8, comprises a mask adapted to seal around a nose and/or mouth of the user to deliver filtered air to the user, wherein the mask comprises a filtered mask inlet port, an exhalation port, and a tube docking connector integrated into the filtered mask inlet port to allow quick connection and disconnection of the mask end connector of the conduit, and wherein the tube docking connector comprises one or more engagement protrusions that are configured to engage with one or more engagement slots in the locking ring of the mask end connector of the conduit to form a connection between the mask and the conduit.

10. The powered respirator device of claim 9, wherein the mask comprises one of:
a full-face mask covering the eyes, nose and mouth, wherein the full-face mask is configured to seal on a region of the face surrounding the eyes, nose and mouth, and the full-face mask comprises a transparent visor,
a disposable fabric face mask that is adapted to cover at least the nose and mouth of the user, or,
a reusable elastomeric face mask that is adapted to cover at least the nose and mouth and the reusable face mask includes one or more vents formed in a body of the mask to vent exhaled gases; and,
wherein each mask comprises the mask end connector.

11. The powered respirator device of claim 4, wherein the wearable air supply unit comprises a thermoelectric cooling (TEC) device integrated into the bridging member, wherein the TEC device is configured to provide heat dissipation to a user and provide localized cooling.

12. The powered respirator device of claim 11, wherein the TEC device is integrated into the neck band of the wearable air supply unit, wherein the TEC device is positioned to contact a posterior aspect of the user's neck in use,
wherein the TEC device comprises:
a thermoelectric substrate,
a proximal surface adapted to be contact the user's skin in the posterior aspect of the neck,
a distal surface exposed to ambient air,
wherein the TEC device is electrically coupled to the battery, and wherein the TEC module is activated by power from the battery and configured to actively transfer heat from its proximal surface to its distal surface, thereby providing the localized cooling to the user's neck.

13. The powered respirator device of claim 4, comprising at least one sensor operatively coupled to the controller and adapted to transmit a detected parameter to the controller, wherein the sensor is configured to detect at least one of an environmental parameter or a physiological parameter; and wherein the controller is programmed to modulate a speed of the blower in real-time based on data received from the at least one sensor to optimize filtration effectiveness and/or optimize power consumption of the power supply.

14. The powered respirator device of claim 13, wherein the sensor comprises a pressure sensor adapted to measure pressure of the flow of gases from the blower, the pressure sensor configured to determine an inhalation phase or a breathing pattern of a user based on the measured pressure, and the controller further configured to control the blower to provide gases synchronized with the inhalation phase or breathing pattern of the user such that the flow is higher during an inhalation phase as compared to an exhalation phase.

15. The powered respirator device of claim 13, wherein the sensor comprises a particulate sensor adapted to detect the ambient particulate matter, and wherein the controller is configured to: increase blower speed in response to an increase in detected ambient particulate matter or control the blower to a blower speed that is correlated to the measured ambient particulate matter.

16. The powered respirator device of claim 6, wherein the mask comprises: a first frame for covering a user's face, wherein the first frame has a first opening opposite to the lips of the user, a light-transmitting sheet engaging an edge of the first opening to cover the first opening, such that light on an inner side of the first frame can be transmitted to an outer side of the first frame through the light-transmitting sheet, wherein the light-transmitting sheet is convex, the base is arranged on the inner side of the light-transmitting sheet and includes a ventilation channel internally, the light-transmitting sheet including air holes in communication with the ventilation channel.

17. A powered respirator device for providing filtered air comprising:
a wearable air supply unit, the wearable air supply unit comprising a blower, a controller and a filter, wherein the wearable air supply unit is adapted to worn around a user's neck or on a user's shoulders,
a mask comprising: a first frame for covering a user's face, wherein the first frame has a first opening opposite to the lips of the user; a light-transmitting sheet engaging an edge of the first opening to cover the first opening, such that light on an inner side of the first frame can be transmitted to an outer side of the first frame through the light-transmitting sheet, wherein the light-transmitting sheet is convex; and a filter assembly for filtering inhaled and exhaled air, wherein the filter assembly comprises a base and a filter element, the base is arranged on the inner side of the light-transmitting sheet and is a has a ventilation channel internally, the light-transmitting sheet comprises air holes in communication with the ventilation channel, and the filter element is installed in the ventilation channel,
a conduit fluidly coupling the wearable air supply unit to the mask,
wherein the blower is configured to draw ambient air through the filter and deliver a continuous flow of filtered air to the mask via the conduit to maintain a continuous positive pressure within the mask, and wherein the filter is a replaceable filter.

18. The powered respirator device of claim 17, wherein the wearable air supply unit comprises a first housing and a second housing, the first and second housing being spaced apart from each other and connected by a bridging member, the bridging member adapted to be suspended from a user's body to support the wearable air supply unit,
wherein the bridging member is a neck band adapted to be worn around a posterior aspect of a user's neck and wherein the first housing and second housing are configured to rest on a user's chest and/or shoulders to distribute wearable air supply unit's weight across the chest and/or shoulders and/or upper back,
wherein the neck band comprises a substantially U shape or an arcuate shape, and first housing and second housing are identical in size and shape such that the weight of the wearable air supply unit is evenly distributed,
wherein the first housing contains a power supply, wherein the power supply is a battery and the second housing contains the blower and the filter, wherein the filter is a replaceable filter, and the power supply is electrically coupled to the blower to provide power to the blower, and the second housing being spaced away from the mask; and,
wherein the replaceable filter is a HEPA (High Efficiency Particulate Air) filter that is rated to at least a N95 level of filtration and the filter is upstream of the blower.

19. The powered respirator device of claim 18, wherein the conduit is a flexible conduit, the conduit comprises a unit end connector to connect to the wearable air supply unit and a mask end connector adapted to connect to the mask, wherein the mask end comprises, wherein the mask end connector comprises a connector housing, the connector housing defining a gas outlet opening, a sealing ring positioned around the gas outlet opening and a locking ring attached to the connector housing, wherein the locking ring is configured to releasably engage with a corresponding connector on the mask.

20. A wearable air supply unit for a personal respirator, comprising: a first housing containing a power source, a second housing containing a blower and a filter, and a neckband interconnecting the first and second housings,
wherein the neckband configured to suspend the housings from a user's neck such that the weight is distributed across the user's clavicle area,
wherein the second housing includes a filtered air discharge port configured for fluid connection to a flexible conduit; and,
wherein the filter is a removable filter that may be removably attachable to the second housing, the filter being positioned upstream of the blower, and the filter filter is a HEPA (High Efficiency Particulate Air) filter that is rated to at least a N95 level of filtration.
